(19) United States
(12) Patent Application Publication
(10) Pub. No.: US 2009/0036469 A1
(43) Pub. Date: Feb. 5, 2009

(54) 2,6-SUBSTITUTED-4-MONOSUBSTITUTED AMINO-PYRIMIDINE AS PROSTAGLANDIN D2 RECEPTOR ANTAGONISTS

(75) Inventors: David STEFANY, Bridgewater, NJ (US); Keith John HARRIS, Bridgewater, NJ (US); Timothy Alan GILLESPY, Bridgewater, NJ (US); Charles J. GARDNER, Bridgewater, NJ (US); Joacy C. AGUIAR, Bridgewater, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/246,918

(22) Filed: Oct. 7, 2008

Related U.S. Application Data
(63) Continuation of application No. PCT/US2007/066481, filed on Apr. 12, 2007.
(60) Provisional application No. 60/744,676, filed on Apr. 12, 2006.

(51) Int. Cl. C07D 239/47; A61K 31/506; A61K 31/505; C07D 401/04; C07D 409/04; A61K 31/495; A61P 37/08; A61P 17/00; A61P 11/00

(52) U.S. Cl. 514/255.04; 544/317; 514/274

(57) ABSTRACT

The present invention is directed to a compound of formula (I),

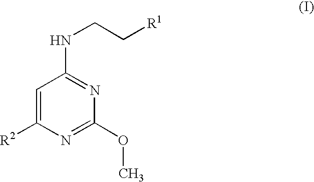

wherein R¹ and R² are as defined herein, a pharmaceutical composition comprising a pharmaceutically effective amount of one or more compounds of the invention in admixture with a pharmaceutically acceptable carrier, and a method of treating a patient suffering from a PGD2-mediated disorder by administering to said patient a pharmaceutically effective amount of a compound of the invention.

### FIELD OF THE INVENTION

The present invention is directed to 2,6-substituted-4-monosubstitutedamino-pyrimidine compounds, their preparation, pharmaceutical compositions containing these compounds, and their pharmaceutical use in the treatment of disease states capable of being modulated by the inhibition of the prostaglandin D2 receptor.

### BACKGROUND OF THE INVENTION

Local allergen challenge in patients with allergic rhinitis, bronchial asthma, allergic conjunctivitis and atopic dermatitis has been shown to result in rapid elevation of prostaglandin D2 “(PGD2)” levels in nasal and bronchial lavage fluids, tears and skin chamber fluids. PGD2 has many inflammatory actions, such as increasing vascular permeability in the conjunctiva and skin, increasing nasal airway resistance, airway narrowing and eosinophil infiltration into the conjunctiva and trachea.

PGD2 is the major cyclooxygenase product of arachidonic acid produced from mast cells on immunological challenge [Lewis, R A, Soter N A, Diamond P T, Austen K F, Oates J A, Roberts L J II, prostaglandin D2 generation after activation of rat and human mast cells with anti-IgE, J. Immunol. 129, 1627-1631, 1982]. Activated mast cells, a major source of PGD2, are one of the key players in driving the allergic response in conditions such as asthma, allergic rhinitis, allergic conjunctivitis, allergic dermatitis and other diseases [Brightling C E, Bradding P, Pavord I D, Wardlaw A J, New Insights into the role of the mast cell in asthma, Clin Exp Allergy 33, 550-556, 2003].

Many of the actions of PGD2 are mediated through its action on the D-type prostaglandin (“DP”) receptor, a G protein-coupled receptor expressed on epithelium and smooth muscle.

In asthma, the respiratory epithelium has long been recognized as a key source of inflammatory cytokines and chemokines that drive the progression of the disease [Holgate S, Lackie P, Wilson S, Roche W, Davies D, Bronchial Epithelium as a Key Regulator of Airway Allergen Sensitization and Remodeling in Asthma, Am J Respir Crit Care Med. 162, 113-117, 2000]. In an experimental murine model of asthma, the DP receptor is dramatically up-regulated on airway epithelium on antigen challenge [Matsuoka T, Hirata M, Tanaka H, Takahashi Y, Murata T, Kabashima K, Sugimoto Y, Kobayashi T, Ushikubi F, Aze Y, Eguchi N, Urade Y, Yoshida N, Kimura K, Mizoguchi A, Honda Y, Nagai H, Narumiya S, prostaglandin D2 as a mediator of allergic asthma, Science 287, 2013-2017, 2000]. In knockout mice, lacking the DP receptor, there is a marked reduction in airway hypereactivity and chronic inflammation [Matsuoka T, Hirata M, Tanaka H, Takahashi Y, Murata T, Kabashima K, Sugimoto Y, Kobayashi T, Ushikubi F, Aze Y, Eguchi N, Urade Y, Yoshida N, Kimura K, Mizoguchi A, Honda Y, Nagai H, Narumiya S, Prostaglandin D2 as a mediator of allergic asthma, Science 287, 2013-2017, 2000]; two of the cardinal features of human asthma.

The DP receptor is also thought to be involved in human allergic rhinitis, a frequent allergic disease that is characterized by the symptoms of sneezing, itching, rhinorea and nasal congestion. Local administration of PGD2 to the nose causes a dose dependent increase in nasal congestion [Doyle W J, Boehm S, Skoner D P, Physiologic responses to intranasal dose-response challenges with histamine, methacholine, bradykinin, and prostaglandin in adult volunteers with and without nasal allergy, J Allergy Clin Immunol. 86(6 Pt 1), 924-35, 1990].

DP receptor antagonists have been shown to reduce airway inflammation in a guinea pig experimental asthma model [Arimura A, Yasui K, Kishino J, Asanuma F, Hasegawa H, Kakudo S, Ohtani M, Arita H (2001), Prevention of allergic inflammation by a novel prostaglandin receptor antagonist, S-5751, J Pharmacol Exp Ther. 298(2), 411-9, 2001]. PGD2, therefore, appears to act on the DP receptor and plays an important role in elicitation of certain key features of allergic asthma.

DP antagonists have been shown to be effective at alleviating the symptoms of allergic rhinitis in multiple species, and more specifically have been shown to inhibit the antigen-induced nasal congestion, the most manifest symptom of allergic rhinitis [Jones, T. R., Savoie, C., Robichaud, A., Sturino, C., Scheigetz, J., Lachance, N., Roy, B., Boyd, M., Abraham, W., Studies with a DP receptor antagonist in sheep and guinea pig models of allergic rhinitis, Am. J. Resp. Crit. Care Med. 167, A218, 2003; and Arimura A, Yasui K, Kishino J, Asanuma F, Hasegawa H, Kakudo S, Ohtani M, Arita H Prevention of allergic inflammation by a novel prostaglandin receptor antagonist, S-5751. J Pharmacol Exp Ther. 298(2), 411-9, 2001].

DP antagonists are also effective in experimental models of allergic conjunctivitis and allergic dermatitis [Arimura A, Yasui K, Kishino J, Asanuma F, Hasegawa H, Kakudo S, Ohtani M, Arita H, Prevention of allergic inflammation by a novel prostaglandin receptor antagonist, S-5751. J Pharmacol Exp Ther. 298(2), 411-9, 2001; and Torisu K, Kobayashi K, Iwahashi M, Nakai Y, Onoda T, Nagase T, Sugimoto I, Okada Y, Matsumoto R, Nanbu F, Ohuchida S, Nakai H, Toda M, Discovery of a new class of potent, selective, and orally active prostaglandin D₂ receptor antagonists, Bioorg. & Med. Chem. 12, 5361-5378, 2004].

### SUMMARY OF THE INVENTION

The present invention is directed to a compound of formula (I):

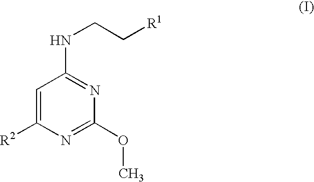

wherein:
R¹ is 2,4-dichloro-phenyl or 4-trifluoromethoxy-phenyl, and
when R¹ is 2,4-dichloro-phenyl, then R² is 3-carboxy-pyrrolidinyl, 3,5-di-(1-hydroxy-1-methyl-ethyl)-phenyl, 3-amino-piperidin-1-yl, 4-amino-piperidin-1-yl, 4-acetamide-piperidin-1-yl, 1-methyl-2-carboxy-2,3-dihydro-1H-indol-5-yl, 3-(1-tert-butylsulfonylaminocarbonyl-1-methyl-ethyl)-phenyl, 3-(1-dimethylaminosulfonylaminocarbonyl-1-methyl-ethyl)-phenyl, 3-(1-thiomorpholin-4-ylcarbonyl-1-methyl-ethyl)-phenyl, 3-(1-aminocarbonyl-1-methyl-ethyl)-phenyl, 3-(1-dimethylaminocarbonyl-1-methyl-ethyl)-phenyl, 3-carboxymethyl-piperidin-1-yl, 3-methylsulfonylaminocarbonyl-piperidin-1-yl, 3-ethylsulfonylaminocarbonyl-piperidin-1-yl, 3-tert-butylsulfonylaminocarbonyl-piperidin-1-yl, 3-trifluoromethylsulfonylaminocarbonyl-piperidin-1-yl, 3-[(1H-tetrazol-5-yl)-aminocarbonyl]-piperidin-1-yl, 3-aminocarbonyl-piperidin-1-yl, 3-dimethylaminocarbonyl-piperidin-1-yl, 3-dimethylaminosulfonylaminocarbonyl-piperidin-1-yl, or 2-carboxy-2,3-dihydro-benzofuran-5-yl, and
when R¹ is 4-trifluoromethoxy-phenyl, then R² is 3-(1-methyl-1-carboxy-ethyl)-piperidinyl, 3-carboxy-piperidinyl, 3-methylsulfonylaminocarbonyl-piperidin-1-yl, 5-carboxy-thiophen-2-yl,
   or a pharmaceutically acceptable salt, hydrate, or solvate thereof, a pharmaceutically acceptable prodrug thereof, or a pharmaceutically acceptable salt, hydrate or solvate of the prodrug.

Another aspect of the present invention is a pharmaceutical composition comprising, a pharmaceutically effective amount of one or more compounds according to the invention, or a pharmaceutically acceptable salt, hydrate, or solvate thereof, a pharmaceutically acceptable prodrug thereof, or a pharmaceutically acceptable salt, hydrate or solvate of the prodrug, in admixture with a pharmaceutically acceptable carrier.

Another aspect of the present invention is a method of treating a patient suffering from a PGD2-mediated disorder including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, retinal detachment, inflammation, infection and sleeping disorders) which are generated secondarily as a result of behavior accompanied by itch (such as scratching and beating), inflammation, chronic obstructive pulmonary diseases, ischemic reperfusion injury, cerebrovascular accident, chronic rheumatoid arthritis, pleurisy, ulcerative colitis and the like by administering to said patient a pharmaceutically effective amount of a compound according to the invention, or a pharmaceutically acceptable salt, hydrate, or solvate thereof, a pharmaceutically acceptable prodrug thereof, or a pharmaceutically acceptable salt, hydrate or solvate of the prodrug.

### DETAILED DESCRIPTION OF THE INVENTION

### Definition of the Terms

As used above, and throughout the description of the invention, the following terms, unless otherwise indicated, shall be understood to have the following meanings:

“Compounds of the present invention”, and equivalent expressions, are meant to embrace compounds of formula (I) as described herein, which expression includes the pharmaceutically acceptable salts, the solvates, e.g., hydrates, the prodrugs, and the pharmaceutically acceptable salts, solvates and hydrates of the prodrugs where the context so permits. Similarly, reference to intermediates, whether or not they themselves are claimed, is meant to embrace their salts, and solvates, where the context so permits.

“Patient” includes human and other mammals.

“Pharmaceutically acceptable salts” refers to the non-toxic, inorganic and organic acid addition salts, and base addition salts, of compounds of the present invention. These salts can be prepared in situ during the final isolation and purification of the compounds.

“Pharmaceutically effective amount” means an amount of compound or compounds according to the present invention effective that produces the desired therapeutic effect described herein, such as allergy relieving, or inflammatory relieving effect.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” refers to compounds that are transformed in vivo to yield a parent compound of the present invention, for example by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propanoyl, butanoyl, and the like), unsubstituted and substituted aroyl (such as benzoyl and substituted benzoyl), alkoxycarbonyl (such as ethoxycarbonyl), trialkylsilyl (such as trimethyl and triethylsilyl), and monoesters formed with dicarboxylic acids (such as succinyl). Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

“Ester prodrug” means a compound that is convertible in vivo by metabolic means (e.g., by hydrolysis) to a compound of the invention. For example an ester of a compound of the invention containing a hydroxy group may be convertible by hydrolysis in vivo to the parent molecule. Alternatively an ester of a compound of the invention containing a carboxy group may be convertible by hydrolysis in vivo to the parent molecule. Exemplary ester prodrugs are:

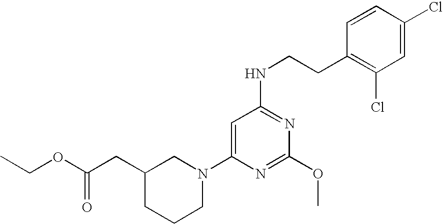

(1-{6-[2-(2,4-Dichloro-phenyl)-ethylamino]-2-methoxy-pyrimidin-4-yl}-piperidin-3-yl)-acetic acid ethyl ester; and

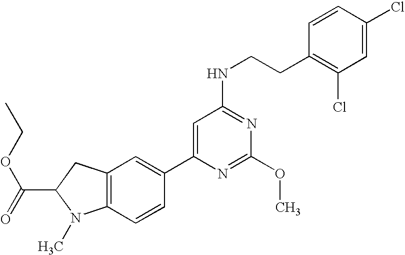

5-{6-[2-(2,4-Dichloro-phenyl)-ethylamino]-2-methoxy-pyrimidin-4-yl}-1-methyl-2,3-dihydro-1H-indole-2-carboxylic acid ethyl ester.

Suitable esters of compounds of the invention containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-para-toluoyltartrates, methanesulfonates, ethanesulfonates, benzenesulfonates, para-toluenesulfonates, cyclohexylsulfamates and quinates.

Suitable esters of compounds of the invention containing a carboxy group, are for example those described by F. J. Leinweber, Drug Metab. Res., 1987, 18, page 379.

An especially useful class of esters of compounds of the invention containing a hydroxy group, may be formed from acid moieties selected from those described by Bundgaard et. al., J. Med. Chem., 1989, 32, pages 2503-2507, and include substituted (aminomethyl)-benzoates, for example dialkylamino-methylbenzoates in which the two alkyl groups may be joined together and/or interrupted by an oxygen atom or by an optionally substituted nitrogen atom, e.g., an alkylated nitrogen atom, more especially (morpholino-methyl)benzoates, e.g., 3- or 4-(morpholinomethyl)-benzoates, and (4-alkylpiperazin-1-yl)benzoates, e.g., 3- or 4-(4-alkylpiperazin-1-yl)benzoates.

“Solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Some of the compounds of the present invention are basic, and such compounds are useful in the form of the free base, or in the form of a pharmaceutically acceptable acid addition salt thereof.

Acid addition salts are a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. In particular, acid addition salts can be prepared by separately reacting the purified compound in its free base form with a suitable organic or inorganic acid and isolating the salt thus formed. Pharmaceutically acceptable salts within the scope of the invention include those derived from mineral acids and organic acids. Exemplary acid addition salts include the hydrobromide, hydrochloride, sulfate, bisulfate, phosphate, nitrate, acetate, oxalate, valerate, oleate, palmitate, quinates, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-para-toluoyltartrates, ethanesulfonates, benzenesulfonates, cyclohexylsulfamates and laurylsulfonate salts. See, for example S. M. Berge, et al., “Pharmaceutical Salts,” J. Pharm. Sci., 66, 1-19 (1977), that is incorporated herein by reference.

Where the compound of the invention is substituted with an acidic moiety, base addition salts may be formed and are simply a more convenient form for use; and in practice, use of the salt form inherently amounts to use of the free acid form. The bases which can be used to prepare the base addition salts include preferably those which produce, when combined with the free acid, pharmaceutically acceptable salts, that is, salts whose cations are non-toxic to the patient in pharmaceutical doses of the salts, so that the beneficial inhibitory effects inherent in the free base are not vitiated by side effects ascribable to the cations. Base addition salts can also be prepared by separately reacting the purified compound in its acid form with a suitable organic or inorganic base derived from alkali and alkaline earth metal salts and isolating the salt thus formed. Base addition salts include pharmaceutically acceptable metal and amine salts. Suitable metal salts include the sodium, potassium, calcium, barium, zinc, magnesium, and aluminum salts. Particular salts are the sodium and potassium salts. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine.

As well as being useful in themselves as active compounds, salts of compounds of the invention are useful for the purposes of purification of the compounds, for example by exploitation of the solubility differences between the salts and the parent compounds, side products and/or starting materials by techniques well known to those skilled in the art.

It will be appreciated that compounds of the present invention may contain asymmetric centers. These asymmetric centers may independently be in either the R or S configuration. It will be apparent to those skilled in the art that certain compounds of the invention may also exhibit geometrical isomerism. It is to be understood that the present invention includes individual geometrical isomers and stereoisomers and mixtures thereof, including racemic mixtures, of compounds of the invention hereinabove. Such isomers can be separated from their mixtures, by the application or adaptation of known methods, for example chromatographic techniques and recrystallization techniques, or they are separately prepared from the appropriate isomers of their intermediates. Additionally, in situations where tautomers of the compounds of the invention are possible, the present invention is intended to include all tautomeric forms of the compounds.

### PARTICULAR EMBODIMENTS OF THE INVENTION

One particular embodiment of the invention is a compound of formula (I), which is
1-{6-[2-(2,4-Dichloro-phenyl)-ethylamino]-2-methyl-pyrimidin-4-yl}-pyrrolidine-3-carboxylic acid,
2-(1-{2-Methoxy-6-[2-(4-trifluoromethoxy-phenyl)-ethylamino]-pyrimidin-4-yl}-piperidin-3-yl)-2-methyl-propionic acid,
2-[3-{6-[2-(2,4-Dichloro-phenyl)-ethylamino]-2-methoxy-pyrimidin-4-yl}-5-(1-hydroxy-1-methyl-ethyl)-phenyl]-propan-2-ol,
[6-(3-Amino-piperidin-1-yl)-2-methoxy-pyrimidin-4-yl]-[2-(2,4-dichloro-phenyl)-ethyl]-amine,
[6-(4-Amino-piperidin-1-yl)-2-methoxy-pyrimidin-4-yl]-[2-(2,4-dichloro-phenyl)-ethyl]-amine,
N-(1-{6-[2-(2,4-Dichloro-phenyl)-ethylamino]-2-methoxy-pyrimidin-4-yl}-piperidin-4-yl)-acetamide,
5-{6-[2-(2,4-Dichloro-phenyl)-ethylamino]-2-methoxy-pyrimidin-4-yl}-1-methyl-2,3-dihydro-1H-indole-2-carboxylic acid,
2-Methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide,
N,N-dimethylamide-2-sulfonic acid [2-(3-{6-[2-(2,4-dichloro-phenyl)-ethylamino]-2-methoxy-pyrimidin-4-yl}-phenyl)-2-methyl-propionyl]-amide,
2-(3-{6-[2-(2,4-Dichloro-phenyl)-ethylamino]-2-methoxy-pyrimidin-4-yl}-phenyl)-2-methyl-1-thiomorpholin-4-yl-propan-1-one,
2-(3-{6-[2-(2,4-Dichloro-phenyl)-ethylamino]-2-methoxy-pyrimidin-4-yl}-phenyl)-isobutyramide,
2-(3-{6-[2-(2,4-Dichloro-phenyl)-ethylamino]-2-methoxy-pyrimidin-4-yl}-phenyl)-N,N-dimethyl-isobutyramide,
(1-{6-[2-(2,4-Dichloro-phenyl)-ethylamino]-2-methoxy-pyrimidin-4-yl}-piperidin-3-yl)-acetic acid,
1-{2-Methoxy-6-[2-(4-trifluoromethoxy-phenyl)-ethylamino]-pyrimidin-4-yl}-piperidine-3-carboxylic acid,
N-(1-{2-Methoxy-6-[2-(4-trifluoromethoxy-phenyl)-ethylamino]-pyrimidin-4-yl}-piperidine-3-carbonyl)-methanesulfonamide,
N-(1-{6-[2-(2,4-Dichloro-phenyl)-ethylamino]-2-methoxy-pyrimidin-4-yl}-piperidine-3-carbonyl)-methanesulfonamide,
Ethanesulfonic acid (1-{6-[2-(2,4-dichloro-phenyl)-ethylamino]-2-methoxy-pyrimidin-4-yl}-piperidine-3-carbonyl)-amide,
2-Methyl-propane-2-sulfonic acid (1-{6-[2-(2,4-dichloro-phenyl)-ethylamino]-2-methoxy-pyrimidin-4-yl}-piperidine-3-carbonyl)-amide,
N-(1-{6-[2-(2,4-Dichloro-phenyl)-ethylamino]-2-methoxy-pyrimidin-4-yl}-piperidine-3-carbonyl)-C,C,C-trifluoro-methanesulfonamide,
1-{6-[2-(2,4-Dichloro-phenyl)-ethylamino]-2-methoxy-pyrimidin-4-yl}-piperidine-3-carboxylic acid (1H-tetrazol-5-yl)-amide,
1-{6-[2-(2,4-Dichloro-phenyl)-ethylamino]-2-methoxy-pyrimidin-4-yl}-piperidine-3-carboxylic acid amide,
1-{6-[2-(2,4-Dichloro-phenyl)-ethylamino]-2-methoxy-pyrimidin-4-yl}-piperidine-3-carboxylic acid dimethylamide,
N,N-Dimethylamide-2-sulfonic acid 1-{6-[2-(2,4-dichloro-phenyl)-ethylamino]-2-methoxy-pyrimidin-4-yl}-piperidine-3-carboxamide,
5-{2-Methoxy-6-[2-(4-trifluoromethoxy-phenyl)-ethylamino]-pyrimidin-4-yl}-thiophene-2-carboxylic acid, or
5-{6-[2-(2,4-Dichloro-phenyl)-ethylamino]-2-methoxy-pyrimidin-4-yl}-2,3-dihydro-benzofuran-2-carboxylic acid,
   or a pharmaceutically acceptable salt, hydrate, or solvate thereof, a pharmaceutically acceptable prodrug thereof, or a pharmaceutically acceptable salt, hydrate or solvate of the prodrug.

Another particular embodiment of the invention is the compound of formula (I) or an ester prodrug thereof, which is
1-{6-[2-(2,4-Dichloro-phenyl)-ethylamino]-2-methyl-pyrimidin-4-yl}-pyrrolidine-3-carboxylic acid,
2-(1-{2-Methoxy-6-[2-(4-trifluoromethoxy-phenyl)-ethylamino]-pyrimidin-4-yl}-piperidin-3-yl)-2-methyl-propionic acid,
2-[3-{6-[2-(2,4-Dichloro-phenyl)-ethylamino]-2-methoxy-pyrimidin-4-yl}-5-(1-hydroxy-1-methyl-ethyl)-phenyl]-propan-2-ol,
[6-(3-Amino-piperidin-1-yl)-2-methoxy-pyrimidin-4-yl]-[2-(2,4-dichloro-phenyl)-ethyl]-amine,
[6-(4-Amino-piperidin-1-yl)-2-methoxy-pyrimidin-4-yl]-[2-(2,4-dichloro-phenyl)-ethyl]-amine,
N-(1-{6-[2-(2,4-Dichloro-phenyl)-ethylamino]-2-methoxy-pyrimidin-4-yl}-piperidin-4-yl)-acetamide,
5-{6-[2-(2,4-Dichloro-phenyl)-ethylamino]-2-methoxy-pyrimidin-4-yl}-1-methyl-2,3-dihydro-1H-indole-2-carboxylic acid,
2-Methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide,
N,N-dimethylamide-2-sulfonic acid [2-(3-{6-[2-(2,4-dichloro-phenyl)-ethylamino]-2-methoxy-pyrimidin-4-yl}-phenyl)-2-methyl-propionyl]-amide,
2-(3-{6-[2-(2,4-Dichloro-phenyl)-ethylamino]-2-methoxy-pyrimidin-4-yl}-phenyl)-2-methyl-1-thiomorpholin-4-yl-propan-1-one,
2-(3-{6-[2-(2,4-Dichloro-phenyl)-ethylamino]-2-methoxy-pyrimidin-4-yl}-phenyl)-isobutyramide,
2-(3-{6-[2-(2,4-Dichloro-phenyl)-ethylamino]-2-methoxy-pyrimidin-4-yl}-phenyl)-N,N-dimethyl-isobutyramide,
(1-{6-[2-(2,4-Dichloro-phenyl)-ethylamino]-2-methoxy-pyrimidin-4-yl}-piperidin-3-yl)-acetic acid,
1-{2-Methoxy-6-[2-(4-trifluoromethoxy-phenyl)-ethylamino]-pyrimidin-4-yl}-piperidine-3-carboxylic acid,
N-(1-{2-Methoxy-6-[2-(4-trifluoromethoxy-phenyl)-ethylamino]-pyrimidin-4-yl}-piperidine-3-carbonyl)-methanesulfonamide,
5-{6-[2-(2,4-dichloro-phenyl)-ethylamino]-2-methoxy-pyrimidin-4-yl}-1-methyl-2,3-dihydro-1H-indole-2-carboxylic acid ethyl ester,
(1-{6-[2-(2,4-dichloro-phenyl)-ethylamino]-2-methoxy-pyrimidin-4-yl}-piperidin-3-yl)-acetic acid ethyl ester,
N-(1-{6-[2-(2,4-Dichloro-phenyl)-ethylamino]-2-methoxy-pyrimidin-4-yl}-piperidine-3-carbonyl)-methanesulfonamide,
Ethanesulfonic acid (1-{6-[2-(2,4-dichloro-phenyl)-ethylamino]-2-methoxy-pyrimidin-4-yl}-piperidine-3-carbonyl)-amide,
2-Methyl-propane-2-sulfonic acid (1-{6-[2-(2,4-dichloro-phenyl)-ethylamino]-2-methoxy-pyrimidin-4-yl}-piperidine-3-carbonyl)-amide,
N-(1-{6-[2-(2,4-Dichloro-phenyl)-ethylamino]-2-methoxy-pyrimidin-4-yl}-piperidine-3-carbonyl)-C,C,C-trifluoro-methanesulfonamide,
1-{6-[2-(2,4-Dichloro-phenyl)-ethylamino]-2-methoxy-pyrimidin-4-yl}-piperidine-3-carboxylic acid (1H-tetrazol-5-yl)-amide,
1-{6-[2-(2,4-Dichloro-phenyl)-ethylamino]-2-methoxy-pyrimidin-4-yl}-piperidine-3-carboxylic acid amide,
1-{6-[2-(2,4-Dichloro-phenyl)-ethylamino]-2-methoxy-pyrimidin-4-yl}-piperidine-3-carboxylic acid dimethylamide,
N,N-Dimethylamide-2-sulfonic acid 1-{6-[2-(2,4-dichloro-phenyl)-ethylamino]-2-methoxy-pyrimidin-4-yl}-piperidine-3-carboxamide,
5-{2-Methoxy-6-[2-(4-trifluoromethoxy-phenyl)-ethylamino]-pyrimidin-4-yl}-thiophene-2-carboxylic acid, or
5-{6-[2-(2,4-Dichloro-phenyl)-ethylamino]-2-methoxy-pyrimidin-4-yl}-2,3-dihydro-benzofuran-2-carboxylic acid,
   or a pharmaceutically acceptable salt, hydrate, or solvate thereof.

The compounds of present invention and the intermediates and starting materials used in their preparation are named in accordance with IUPAC rules of nomenclature in which the characteristic groups have decreasing priority for citation as the principle group as follows: acids, esters, amides, etc. However, it is understood that, for a particular compound referred to by both a structural Formula and a nomenclature name, if the structural Formula and the nomenclature name are inconsistent with each other, the structural Formula takes the precedence over the nomenclature name.

The compounds of the invention exhibit prostaglandin D2 receptor antagonist activity and are useful as pharmacological acting agents. Accordingly, they are incorporated into pharmaceutical compositions and used in the treatment of patients suffering from certain medical disorders.

Compounds within the scope of the present invention are antagonists of the prostaglandin D2 receptor, according to tests described in the literature and described in pharmacological testing section hereinafter, and which tests results are believed to correlate to pharmacological activity in humans and other mammals. Thus, in a further embodiment, the present invention provides compounds of the invention and compositions containing compounds of the invention for use in the treatment of a patient suffering from, or subject to, conditions, which can be ameliorated by the administration of a PGD2 antagonist. For example, compounds of the present invention could therefore be useful in the treatment of a variety of PGD2-mediated disorders including, but not limited to, allergic disease (such as allergic rhinitis, allergic conjunctivitis, atopic dermatitis, bronchial asthma and food allergy), systemic mastocytosis, disorders accompanied by systemic mast cell activation, anaphylaxis shock, bronchoconstriction, bronchitis, urticaria, eczema, diseases accompanied by itch (such as atopic dermatitis and urticaria), diseases (such as cataract, retinal detachment, inflammation, infection and sleeping disorders) which are generated secondarily as a result of behavior accompanied by itch (such as scratching and beating), inflammation, chronic obstructive pulmonary diseases, ischemic reperfusion injury, cerebrovascular accident, chronic rheumatoid arthritis, pleurisy, ulcerative colitis and the like.

Compounds of the present invention are further useful in treatments involving a combination therapy with:

(i) antihistamines, such as fexofenadine, loratadine and citirizine, for the treatment of allergic rhinitis;
(ii) leukotriene antagonists, such as montelukast and zafirlukast, for the treatment of allergic rhinitis, COPD, allergic dermatitis, allergic conjunctivitis, etc.—please specifically refer to the claims in WO 01/78697 A2;
(iii) beta agonists, such as albuterol, salbuterol and terbutaline, for the treatment of asthma, COPD, allergic dermatitis, allergic conjunctivitis, etc;
(iv) antihistamines, such as fexofenadine, loratadine and citirizine, for the treatment of asthma, COPD, allergic dermatitis, allergic conjunctivitis, etc;
(v) PDE4 (Phosphodiesterase 4) inhibitors, such as roflumilast and cilomilast, for the treatment of asthma, COPD, allergic dermatitis, allergic conjunctivitis, etc; or
(vi) with TP (Thromboxane A2 receptor) or CrTh2 (chemoattractant receptor-homologous molecule expressed on Th2 cells) antagonists, such as Ramatrobran (BAY-u3405), for the treatment of COPD, allergic dermatitis, allergic conjunctivitis, etc.

A special embodiment of the therapeutic methods of the present invention is the treating of allergic rhinitis.

Another special embodiment of the therapeutic methods of the present invention is the treating of bronchial asthma.

According to a further feature of the invention there is provided a method for the treatment of a human, or animal patient suffering from, or subject to, conditions which can be ameliorated by the administration of a prostaglandin D2 receptor antagonist, for example conditions as hereinbefore described, which comprises the administration to the patient of an effective amount of compound of the invention or a composition containing a compound of the invention. “Effective amount” is meant to describe an amount of compound of the present invention effective as a prostaglandin D2 receptor antagonist and thus producing the desired therapeutic effect.

References herein to treatment should be understood to include prophylactic therapy as well as treatment of established conditions.

The present invention also includes within its scope pharmaceutical compositions comprising at least one of the compounds of the invention in admixture with a pharmaceutically acceptable carrier.

In practice, the compound of the present invention may be administered in pharmaceutically acceptable dosage form to humans and other animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

A particular aspect of the invention provides for a compound according to the present invention to be administered in the form of a pharmaceutical composition. Pharmaceutical compositions, according to the present invention, comprise compounds of the present invention and pharmaceutically acceptable carriers.

Pharmaceutically acceptable carriers include at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms.

Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances.

Exemplary antibacterial and antifungal agents for the prevention of the action of microorganisms include parabens, chlorobutanol, phenol, sorbic acid, and the like.

Exemplary isotonic agents include sugars, sodium chloride, and the like.

Exemplary adsorption delaying agents to prolong absorption include aluminum monostearate and gelatin.

Exemplary adsorption promoting agents to enhance absorption include dimethyl sulfoxide and related analogs.

Exemplary diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween®60, Span®60, cetostearyl alcohol, myristyl alcohol, glyceryl mono-stearate and sodium lauryl sulfate, fatty acid esters of sorbitan, vegetable oils (such as cottonseed oil, groundnut oil, com germ oil, olive oil, castor oil and sesame oil) and injectable organic esters such as ethyl oleate, and the like, or suitable mixtures of these substances.

Exemplary excipients include lactose, milk sugar, sodium citrate, calcium carbonate and dicalcium phosphate.

Exemplary disintegrating agents include starch, alginic acids and certain complex silicates.

Exemplary lubricants include magnesium stearate, sodium lauryl sulfate, talc, as well as high molecular weight polyethylene glycols.

The choice of pharmaceutical acceptable carrier is generally determined in accordance with the chemical properties of the active compound such as solubility, the particular mode of administration and the provisions to be observed in pharmaceutical practice.

Pharmaceutical compositions of the present invention suitable for oral administration may be presented as discrete units such as a solid dosage form, such as capsules, cachets or tablets each containing a predetermined amount of the active ingredient, or as a powder or granules; as a liquid dosage form such as a solution or a suspension in an aqueous liquid or a non-aqueous liquid, or as an oil-in-water liquid emulsion or a water-in-oil liquid emulsion. The active ingredient may also be presented as a bolus, electuary or paste.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and Na₂CO₃, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention in a certain part of the intestinal tract in a delayed manner.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used. A mixture of the powdered compounds moistened with an inert liquid diluent may be molded in a suitable machine to make molded tablets. The tablets may optionally be coated or scored and may be formulated so as to provide slow or controlled release of the active ingredient therein. Solid compositions may also be employed as fillers in soft and hard-filled gelatin capsules using such excipients as lactose or milk sugar as well as high molecular weight polyethylene glycols, and the like.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria-retaining filter, or by incorporating sterilizing agents in the form of sterile solid compositions that can be dissolved in sterile water, or some other sterile injectable medium immediately before use.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

When aqueous suspensions are used they can contain emulsifying agents or agents which facilitate suspension.

Pharmaceutical compositions suitable for topical administration mean formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxy groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a composition is based on achieving the desired properties. Thus a cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Pharmaceutical compositions suitable for rectal or vaginal administrations means formulations that are in a form suitable to be administered rectally or vaginally to a patient and containing at least one compound of the invention. Suppositories are a particular form for such formulations that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

Pharmaceutical composition administered by injection may be by transmuscular, intravenous, intraperitoneal, and/or subcutaneous injection. The compositions of the present invention are formulated in liquid solutions, in particular in physiologically compatible buffers such as Hank's solution or Ringer's solution. In addition, the compositions may be formulated in solid form and redissolved or suspended immediately prior to use. Lyophilized forms are also included. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

Pharmaceutical composition of the present invention suitable for nasal or inhalational administration means compositions that are in a form suitable to be administered nasally or by inhalation to a patient. The composition may contain a carrier, in a powder form, having a particle size for example in the range 1 to 500 microns (including particle sizes in a range between 20 and 500 microns in increments of 5 microns such as 30 microns, 35 microns, etc.). Suitable compositions wherein the carrier is a liquid, for administration as for example a nasal spray or as nasal drops, include aqueous or oily solutions of the active ingredient. Compositions suitable for aerosol administration may be prepared according to conventional methods and may be delivered with other therapeutic agents. Metered dose inhalers are useful for administering compositions according to the invention for an inhalational therapy.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses may be in amounts, for example, of from about 0.001 to about 100 mg/kg body weight daily and preferably 0.01 to 10 mg/kg/day. For example, in an adult, the doses are generally from about 0.01 to about 100, preferably about 0.01 to about 10, mg/kg body weight per day by inhalation, from about 0.01 to about 100, preferably 0.1 to 70, more especially 0.5 to 10, mg/kg body weight per day by oral administration, and from about 0.01 to about 50, preferably 0.01 to 10, mg/kg body weight per day by intravenous administration. The percentage of active ingredient in a composition may be varied, though it should constitute a proportion such that a suitable dosage shall be obtained. Dosage unit compositions may contain such amounts of such submultiples thereof as may be used to make up the daily dose. Obviously, several unit dosage forms may be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compounds of the invention may be prepared by the application or adaptation of known methods, by which is meant methods used heretofore or described in the literature, for example those described by R. C. Larock in Comprehensive Organic Transformations, VCH publishers, 1989.

According to a further feature of the invention, acid addition salts of the compounds of this invention may be prepared by reaction of the free base with the appropriate acid, by the application or adaptation of known methods. For example, the acid addition salts of the compounds of this invention may be prepared either by dissolving the free base in water or aqueous alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds of this invention can be regenerated from the salts by the application or adaptation of known methods. For example, parent compounds of the invention can be regenerated from their acid addition salts by treatment with an alkali, e.g. aqueous sodium bicarbonate solution or aqueous ammonia solution.

Compounds of this invention can be regenerated from their base addition salts by the application or adaptation of known methods. For example, parent compounds of the invention can be regenerated from their base addition salts by treatment with an acid, e.g. hydrochloric acid.

Compounds of the present invention may be conveniently prepared or formed during the process of the invention, as solvates (e.g. hydrates). Hydrates of compounds of the present invention may be conveniently prepared by recrystallization from an aqueous/organic solvent mixture, using organic solvents such as dioxane, THF or MeOH.

According to a further feature of the invention, base addition salts of the compounds of this invention may be prepared by reaction of the free acid with the appropriate base, by the application or adaptation of known methods. For example, the base addition salts of the compounds of this invention may be prepared either by dissolving the free acid in water or aqueous alcohol solution or other suitable solvents containing the appropriate base and isolating the salt by evaporating the solution, or by reacting the free acid and base in an organic solvent, in which case the salt separates directly or can be obtained by concentration of the solution.

The starting materials and intermediates may be prepared by the methods described in the present application or adaptation of known methods.

The compounds of the invention, their methods or preparation and their biological activity will appear more clearly from the examination of the following examples that are presented as an illustration only and are not to be considered as limiting the invention in its scope. Compounds of the invention are identified, for example, by the following analytical methods.

High Pressure Liquid Chromatography—Mass Spectrometry (LCMS) experiments to determine retention times (R_{T}) and associated mass ions are performed using one of the following methods.

Mass Spectra (MS) are recorded using a Micromass LCT mass spectrometer. The method is positive electrospray ionization, scanning mass m/z from 100 to 1000. Liquid chromatography is performed on a Hewlett Packard 1100 Series Binary Pump & Degasser; stationary phase: Phenomenex Synergi 2μ Hydro-RP 20×4.0 mm column, mobile phase: A=0.1% formic acid (FA) in water, B=0.1% FA in MeCN. Injection volume of 5 μL by CTC Analytical PAL System. Flow is 1 mL/minute. Gradient is 10% B to 90% B in 3 minutes and 90% B to 100% B in 2 minutes. Auxiliary detectors are: Hewlett Packard 1100 Series UV detector, wavelength=220 nm and Sedere SEDEX 75 Evaporative Light Scattering (ELS) detector temperature=46° C., N₂ pressure=4 bar.

300 MHz ¹H nuclear magnetic resonance spectra (NMR) are recorded at ambient temperature using a Varian Mercury (300 MHz) spectrometer with an ASW 5 mm probe. In the NMR chemical shifts (□) are indicated in parts per million (ppm) with reference to tetramethylsilane (TMS) as the internal standard.

As used in the examples and preparations that follow, as well as the rest of the application, the terms used therein shall have the meanings indicated: “kg” refers to kilograms, “g” refers to grams, “mg” refers to milligrams, “μg” refers to micrograms, “mol” refers to moles, “mmol” refers to millimoles, “M” refers to molar, “mM” refers to millimolar, “μM” refers to micromolar, “nM” refers to nanomolar, “L” refers to liters, “mL” or “ml” refers to milliliters, “μL” refers to microliters, “° C.” refers to degrees Celsius, “mp” or “m.p.” refers to melting point, “bp” or “b.p.” refers to boiling point, “mm of Hg” refers to pressure in millimeters of mercury, “cm” refers to centimeters, “nm” refers to nanometers, “abs.” refers to absolute, “conc.” refers to concentrated, “c” refers to concentration in g/mL, “rt” refers to room temperature, “TLC” refers to thin layer chromatography, “HPLC” refers to high performance liquid chromatography, “i.p.” refers to intraperitoneally, “i.v.” refers to intravenously, “s”=singlet, “d”=doublet; “t”=triplet; “q”=quartet; “m”=multiplet, “dd”=doublet of doublets; “br”=broad, “LC”=liquid chromatograph, “MS”=mass spectrograph, “ESI/MS”=electrospray ionization/mass spectrograph, “R_{T}”=retention time, “M”=molecular ion, “PSI”=pounds per square inch, “DMSO”=dimethyl sulfoxide, “DMF”=N,N-dimethylformamide, “CDI”=1,1′-carbonyldiimidazole, “DCM” or “CH₂Cl₂”=dichloromethane, “HCl”=hydrochloric acid, “SPA”=Scintillation Proximity Assay, “ATTC”=American Type Culture Collection, “FBS”=Foetal Bovine Serum, “MEM”=Minimal Essential Medium, “CPM”=Counts Per Minute, “EtOAc”=ethyl acetate, “PBS”=Phosphate Buffered Saline, “TMD”=transmembrane domain, “IBMX”=3-isobutyl-1-methylxanthine, “cAMP”=cyclic adenosine monophosphate, “IUPAC”=International Union of Pure and Applied Chemistry, “MHz”=megahertz, “PEG”=polyethylene glycol, “MeOH”=methanol, “N”=normality, “THF”=tetrahydrofuran, “h”=hours, “min”=minute(s), “MeNH₂”=methyl amine, “N₂”=nitrogen gas, “O.D.”=outer diameter, “MeCN” or “CH₃CN”=acetonitrile, “Et₂O”=ethyl ether, “Prep LC”=preparatory “flash” liquid chromatography, “SPE”=solid phase extraction, “K₂CO₃”=potassium carbonate, “Na₂CO₃”=sodium carbonate, “pmol”=picomolar, “heptane”=n-heptane, “HMBA-AM” resin=4-hydroxymethylbenzoic acid amino methyl resin, “PdCl₂(dppf)₂”=1,1′-bis(diphenylphosphino)ferrocene-palladium (II) dichloride DCM complex, “˜”=approximately, and “IC₅₀”=concentration of the compound that produces 50% inhibition in the SPA cAMP assay in human LS174 T cells.

### EXAMPLES

### Example 1

### 1-{6-[2-(2,4-Dichloro-phenyl)-ethylamino]-2-methyl-pyrimidin-4-yl}-pyrrolidine-3-carboxylic acid

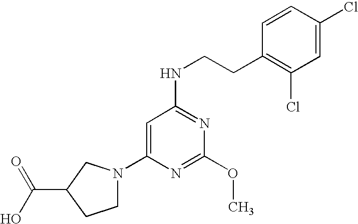

Step 1: A solution of 4,6-dichloro-2-methoxypyrimidine (0.7 g), 2-(2,4-dichloro-phenyl)-ethylamine (0.74 g) and Na₂CO₃ (0.88 g) in EtOH (25 mL) is heated at 80° C. for 3 hours and poured into water (400 mL). The resulting solid is filtered and air dried to afford (6-chloro-2-methoxy-pyrimidin-4-yl)-[2-(2,4-dichloro-phenyl)-ethyl]-amine.

Step 2: In a tube is combined (6-chloro-2-methoxy-pyrimidin-4-yl)-[2-(2,4-dichloro-phenyl)-ethyl]-amine (300 mg), 3-pyrrolidine carboxylic acid hydrochloride (341 mg), K₂CO₃ (373 mg) and 1-methyl-2-pyrrolidinone (5 mL). The tube is sealed and heated to 140° C. and stirred for 16 hours. The mixture is allowed to cool to ambient temperature, diluted with water (60 mL) and acidified using 3M HCl, extracted thrice with ethyl acetate (60 mL). The organic extracts are combined and dried over magnesium sulfate, concentrated, and purified via silica gel chromatography (40 g) eluting with 0 to 20% MeOH in dichloromethane to give 1-{6-[2-(2,4-dichloro-phenyl)-ethylamino]-2-methyl-pyrimidin-4-yl}-pyrrolidine-3-carboxylic acid (190 mg) as a solid. LCMS R_{T}=2.22 minutes, MS: 411 (M+H). ¹H NMR [300 MHz, (CD₃)₂SO]: δ 7.57 (1H, s); 7.36 (2H, s); 6.77 (1H, s); 5.01 (1H, s); 3.72 (3H, s); 3.5 (6H, m); 3.12 (1H, m); 2.91 (2H, t); 2.09 (2H, m). IC₅₀=9 nM.

### Example 2

### 2-(1-{2-Methoxy-6-[2-(4-trifluoromethoxy-phenyl)-ethylamino]-pyrimidin-4-yl}-piperidin-3-yl)-2-methyl-propionic acid

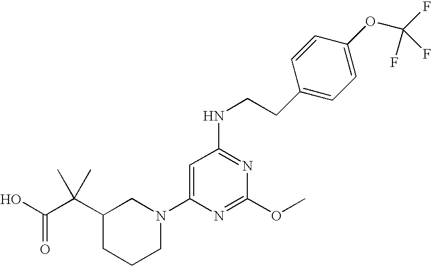

Step 1: A mixture of pyrid-3-ylacetic acid ethyl ester (12.6 g) and rhodium on alumina (12.6 g) in ethanol (200 mL) is put on Parr shaker at 60° C. and 60 PSI for 16 hours. The suspension is filtered through a Celite pad. The pad is washed with ethanol and the filtrate is concentrated to a volume of approximately 50 mL and water (600 mL) is added. The solution is extracted with EtOAc (3×100 mL). The combined organic layer is washed with brine, dried (Na₂SO₄), filtered and evaporated in vacuo. The residue is dissolved in THF (150 mL) and triethylamine (10.7 mL) is added. The solution is cooled to 0° C. and benzylchloroformate (11 mL) is added dropwisely. The solution is stirred at 0° C. for two hours. The solution is concentrated to a volume of approximately 50 mL and water (600 mL) is added. The solution is extracted with EtOAc (2×150 mL). The combined organic layer is washed with brine, dried (Na₂SO₄), filtered and evaporated in vacuo to afford 3-ethoxycarbonylmethyl-piperidine-1-carboxylic acid benzyl ester (21.5 g), which is used for next step without further purification. MS: 306 (M+H); ¹H NMR (300 MHz, DMSO-d₆) δ 7.3 (m, 5H); 5.05 (s, 2H); 3.8-4.1 (m, 4H); 2.5-2.6 (m, 1H); 1.5-1.7 (m, 4H); 1-1.4 (m, 4H).

Step 2: To a 1M suspension of potassium tert-butoxide in THF (200 mL) at −78° C. is added a solution of 3-ethoxycarbonyl-methyl-piperidine-1-carboxylic acid benzyl ester (21.5 g) in THF (25 mL) dropwise over ten minutes. Methyl iodide (6.85 mL) is added in one portion. The suspension is stirred at −78° C. for one hour, at −40° C. for one hour and allowed to warm to room temperature overnight. The suspension is poured into water (800 mL) and extracted with EtOAc (2×150 mL). The combined organic layer is washed with brine, dried (Na₂SO₄), filtered and evaporated in vacuo. The residue is purified by chromatography on silica gel eluting with 100% heptane to 30% EtOAc in heptane to afford 3-(1-ethoxycarbonyl-1-methyl-ethyl)-piperidine-1-carboxylic acid benzyl ester (151.1 g). MS: 334 (M+H); ¹H NMR (300 MHz, DMSO-d₆) δ 7.3 (m, 5H); 5.05 (s, 2H); 3.8-4.1 (q, 2H); 2.5-2.6 (m, 1H); 1.5-1.7 (m, 4H); 1-1.4 (m, 4H); 1 (s, 6H).

Step 3: A suspension of 3-(1-ethoxycarbonyl-1-methyl-ethyl)-piperidine-1-carboxylic acid benzyl ester (3.3 g) and 10% palladium on carbon (500 mg) in glacial acetic acid (2 mL)/methanol (200 mL) is placed on Parr shaker at 50 PSI for 90 minutes at room temperature. The suspension is filtered through a celite pad. The pad is washed with methanol and the filtrate is concentrated to a volume of approximately 50 mL. The methanol solution is diluted with THF (50 mL) and 2N potassium hydroxide aqueous solution (50 mL). The solution is stirred at room temperature for 16 hours, and concentrated to a volume of 70-80 mL in vacuo. The solution is cooled to 5° C. and concentrated aqueous HCl (8.5 mL) is added slowly. The solution is extracted with EtOAc (3×100 mL). The combined organic layer is washed with brine, dried (Na₂SO₄), filtered and evaporated in vacuo to afford 2-methyl-2-piperidin-3-yl-propionic acid (1.1 g), which is used for next step without further purification. MS: 172 (M+H); ¹H NMR (300 MHz, DMSO-d₆) δ 2.5 (m, 1H); 1.5-1.7 (m, 4H); 1-1.4 (m, 5H); 1 (s, 6H).

Step 4: Method A. A solution of (4-trifluoromethoxy-phenyl)-acetonitrile (5.05 g) in MeOH (75 mL) is saturated with ammonia gas, and treated with Raney nickel in water (2 mL, 50%). The suspension is placed on Parr shaker at 50 PSI and 50° C. for 3 hours, and filtered through celite. The filtrate is evaporated and the residual oil is portioned between water and ethyl acetate. The organic phase is dried over sodium sulfate, filtered and evaporated. The residue is dissolved in MeOH and the solution treated with concentrated hydrochloric acid (1 mL) is added. The solution is evaporated in vacuo to a solid which is triturated with ether and air dried to give 2-(4-trifluoromethoxy-phenyl)-ethylamine hydrochloride (5.15 g). MS: 206 (M+H), ¹H NMR (CDCl₃): δ 8.2 (2H, m); 7.4 (2H, d, J=5 Hz); 7.3 (2H, d, J=5 Hz); 3-3.1 (2H, m); 2.9-3 (2H, m).

Method B. A solution of 4-trifluoromethoxy benzaldehyde (1 g) and nitromethane (0.96 g) in acetic acid (10.6 mL) is treated with ammonium acetate (1.01 g) is heated under microwave to 150° C. for 15 minutes. The reaction mixture is diluted with water, and extracted three times with DCM (50 mL). The combined extracts are washed sequentially with 2 N sodium hydroxide, water, and brine, dried over sodium sulfate and concentrated. The residue is subjected to silica gel chromatography to yield 4-trifluoromethoxy-(2-nitro-vinyl)-benzene (1.23 g) as a solid. A portion of 4-trifluoromethoxy-(2-nitro-vinyl)-benzene (0.504 g) is hydrogenated with hydrogen in a balloon, 10% Pd/C (115 mg) in MeOH (22 mL) containing concentrated hydrochloric acid (0.27 mL) at room temperature for 15 hours. The mixture is filtered and filtrate is concentrated to a solid that is washed with Et₂O to obtain 2-(4-trifluoromethoxy-phenyl)-ethylamine hydrochloride (0.3 g) as a solid. LC/MS: MS: 206 (M+H).

Step 5: Proceeding in a similar manner as Example 1, step 1, but using 4,6-dichloro-2-methoxypyrimidine (0.39 g), 2-(4-trifluoromethoxy-phenyl)-ethylamine hydrochloride (0.38) and sodium bicarbonate (0.74 g), there is prepared (6-chloro-2-methoxy-pyrimidin-4-yl)-[2-(4-trifluoromethoxy-phenyl)-ethyl]-amine (0.61 g). MS: 360 (M+H), ¹H NMR (CDCl₃): δ 7.4 (2H, d, J=7 Hz); 7.3 (2H, d, J=7 Hz); 6.2 (1H, s); 3.8 (3H, s); 3.5-3.6 (2H, m); 2.8 (2H, t).

Step 6: A solution of 2-methyl-2-piperidin-3-yl-propionic acid (0.6 g), (6-chloro-2-methoxy-pyrimidin-4-yl)-[2-(4-trifluoromethoxy-phenyl)-ethyl]-amine (0.46 g) and K₂CO₃ (0.46 g) in 1-methylpyrrolidin-2-one (10 mL) is heated at 140° C. for 16 hours. The solution is cooled and poured into water (200 mL). The aqueous solution is acidified to pH 6 with glacial acetic acid and extracted with EtOAc (3×100 mL). The combined organic layer is washed with brine, dried (Na₂SO₄), filtered and evaporated in vacuo. The residue is purified by chromatography on silica gel eluting with 5% MeOH in EtOAc to afford 2-(1-{2-methoxy-6-[2-(4-trifluoromethoxy-phenyl)-ethylamino]-pyrimidin-4-yl}-piperidin-3-yl)-2-methyl-propionic acid (105 mg). MS: 483 (M+H); ¹H NMR (300 MHz, DMSO-d₆) δ 7.45 (d, J=3, 2H); 7.3 (d, J=3, 2H); 5.5 (s, 1H); 3.95 (s, 3H); 3.6 (m, 2H); 2.9 (t, 2H); 2.7 (m, 1H); 1.7-1.9 (m, 4H); 1.3-1.4 (m, 3H); 1.1 (d, J=3, 6H). IC₅₀=2 nM.

### Example 3

### 2-[3-{6-[2-(2,4-Dichloro-phenyl)-ethylamino]-2-methoxy-pyrimidin-4-yl}-5-(1-hydroxy-1-methyl-ethyl)-phenyl]-propan-2-ol

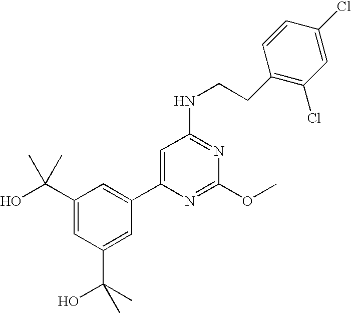

Step 1: A solution of dimethyl-5-bromo isophthalate (5 g) in THF (250 mL) is cooled to −78° C., and a 3 M solution of methyl magnesium bromide in ether (36.6 mL) is added dropwise while maintaining the temperature below −70° C. The solution is stirred at −78° C. for 2 hours and allowed to warm to room temperature overnight. The solution is diluted with ether (300 mL) and cooled to 0° C. 1 N aqueous HCl (100 mL) is added dropwise. The combined organic layer is washed with brine, dried (Na₂SO₄), filtered and evaporated in vacuo. The residue is purified by chromatography on silica gel eluting with 60% EtOAc in heptane to afford 2-[3-bromo-5-(1-hydroxy-1-methyl-ethyl)-phenyl]-propan-2-ol (4.1 g). MS: 272 (M+H); ¹H NMR (300 MHz, DMSO-d₆) δ 7.5 (s, 1H); 7.4 (s, 2H); 5.15 (s, 2H); 1.4 (s, 12H).

Step 2: 2-[3-Bromo-5-(1-hydroxy-1-methyl-ethyl)-phenyl]-propan-2-ol (1.08 g), 4,4,5,5,4′,4′,5′,5′-octamethyl-[2,2′]bi[[1,3,2]-dioxaborolanyl] (1.12 g), potassium acetate (0.78 g) and PdCl₂(dppf)₂ (42 mg) are suspended in DMSO (20 mL) and degassed for 20 minutes. The suspension is heated at 90° C. for 16 hours. The solution is poured into water (300 mL) and extracted with EtOAc (2×150 mL). The combined organic layer is washed with brine, dried (Na₂SO₄), filtered and evaporated in vacuo. The residue is purified by chromatography on silica gel eluting with 50% EtOAc in heptane to afford 2-[3-(1-hydroxy-1-methyl-ethyl)-5-(4,4,5,5-tetramethyl-[1,3,2]dioxaborolan-2-yl)-phenyl]-propan-2-ol (0.9 g). MS: 285 (M+H); ¹H NMR (300 MHz, DMSO-d₆) δ 7.5 (s, 1H); 7.2 (s, 2H); 5.15 (s, 2H); 1.6 (s, 12H); 1.4 (s, 12H).

Step 3: A solution of 2-[3-(1-hydroxy-1-methyl-ethyl)-5-(4,4,5,5-tetramethyl-[1,3,2]dioxaborolan-2-yl)-phenyl]-propan-2-ol (0.35 g), (6-chloro-2-methoxy-pyrimidin-4-yl)-[2-(2,4-dichloro-phenyl)-ethyl]-amine (0.2 g), cesium carbonate (0.58 g) and tetrakis(triphenylphosphine) palladium (0) (41 mg) in 20 mL water/80 mL dimethoxyethane is degassed for 20 minutes and heated at 90° C. for 16 hours. The solution is evaporated in vacuo. The residue is purified by chromatography eluting with 70% EtOAc in heptane to afford to 2-[3-{6-[2-(2,4-dichloro-phenyl)-ethylamino]-2-methoxy-pyrimidin-4-yl}-5-(1-hydroxy-1-methyl-ethyl)-phenyl]-propan-2-ol (0.44 g). MS: 491 (M+H); ¹H NMR (300 MHz, DMSO-d₆) δ 7.9 (s, 2H), 7.8 (s, 1H); 7.45 (s, 1H); 7.2-7.3 (m, 2H); 6.5 (s, 1H); 3.95 (s, 3H); 3.85 (m, 2H); 3.1 (t, 2H); 1.6 (s, 12H). IC₅₀=730 nM.

### Example 4

### [6-(3-Amino-piperidin-1-yl)-2-methoxy-pyrimidin-4-yl]-[2-(2,4-dichloro-phenyl)-ethyl]-amine

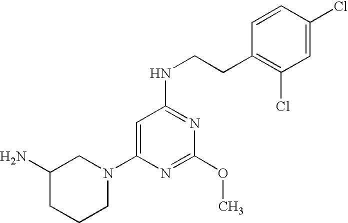

Step 1: By proceeding in a similar manner to Example 1, step 2, but substituting 3-N-Boc-aminopiperidine (450 mg) for 3-pyrrolidine carboxylic acid hydrochloride, and subjecting the reaction product to flash column chromatography on silica gel (40 g) eluting with 20 to 50% EtOAc in heptane there is prepared (1-{6-[2-(2,4-dichloro-phenyl)-ethylamino]-2-methoxy-pyrimidin-4-yl}-piperidin-3-yl)-carbamic acid tert-butyl ester (281 mg). ¹H NMR [300 MHz, (CD₃)₂SO]: δ 7.57 (1H, s); 7.36 (2H, s); 6.9 (2H, m); 5.29 (1H, s); 4 (2H, m); 3.71 (3H, s); 3.41 (5H, m); 2.91 (2H, t); 2.65 (2H, m); 1.82 (1H, s); 1.63 (1H, s); 1.39 (9H, s).

Step 2: A solution of (1-{6-[2-(2,4-Dichloro-phenyl)-ethylamino]-2-methoxy-pyrimidin-4-yl}-piperidin-3-yl)-carbamic acid tert-butyl ester (234 mg) in dichloromethane (4 mL) is treated with trifluoroacetic acid (4 mL). The mixture is stirred at ambient temperature for 3 hours and concentrated in vacuo. The residue is dissolved in saturated sodium bicarbonate solution (25 mL) and extracted twice with ethyl acetate (25 mL). The organic extracts are combined, washed with brine (20 mL), and dried over magnesium sulfate, concentrated, and purified via silica gel chromatography (12 g) eluting with 0 to 10% MeOH in dichloromethane to give [6-(3-amino-piperidin-1-yl)-2-methoxy-pyrimidin-4-yl]-[2-(2,4-dichloro-phenyl)-ethyl]-amine (157 mg) as a solid. LCMS R_{T}=1.77 minutes, MS: 396 (M+H). ¹H NMR [300 MHz, (CD₃)₂SO]: δ 7.59 (1H, s); 7.36 (2H, s); 6.86 (2H, m); 5.93 (1H, b); 5.29 (1H, s); 4.16 (2H, d); 3.82 (2H, d); 3.73 (3H, s); 3.41 (4H, m); 2.91 (4H, m); 1.91 (1H, m); 1.69 (1H, m); 1.41 (2H, m); 1.23 (1H, s). IC₅₀=985 nM.

### Example 5

### (a) [6-(4-Amino-piperidin-1-yl)-2-methoxy-pyrimidin-4-yl]-[2-(2,4-dichloro-phenyl)-ethyl]-amine

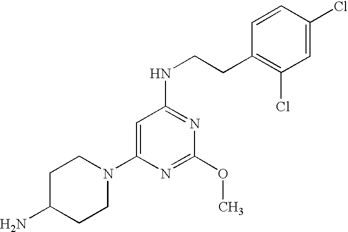

Step 1: By proceeding in a similar manner to Example 1, step 2, but substituting 4-N-Boc-aminopiperidine (450 mg) for 3-pyrrolidine carboxylic acid hydrochloride, and subjecting the reaction product to flash column chromatography on silica gel (40 g) eluting with 0 to 40% EtOAc in heptane there is prepared (1-{6-[2-(2,4-dichloro-phenyl)-ethylamino]-2-methoxy-pyrimidin-4-yl}-piperidin-4-yl)-carbamic acid tert-butyl ester (320 mg).

Step 2: A solution of ((1-{6-[2-(2,4-dichloro-phenyl)-ethylamino]-2-methoxy-pyrimidin-4-yl}-piperidin-4-yl)-carbamic acid tert-butyl ester (300 mg) in DCM (5 mL) is treated with triethylsilane (194 μL) followed by the addition of trifluoroacetic acid (106 μL). The mixture is stirred at ambient temperature for 20 hours and concentrated in vacuo. The residue is dissolved in saturated sodium bicarbonate solution (30 mL) and extracted twice with ethyl acetate (30 mL). The organic extracts are combined, washed with brine (20 mL), and dried over magnesium sulfate, and concentrated to give [6-(3-amino-piperidin-1-yl)-2-methoxy-pyrimidin-4-yl]-[2-(2,4-dichloro-phenyl)-ethyl]-amine (230 mg) as a solid.

### (b) N-(1-{6-[2-(2,4-Dichloro-phenyl)-ethylamino]-2-methoxy-pyrimidin-4-yl}-piperidin-4-yl)-acetamide

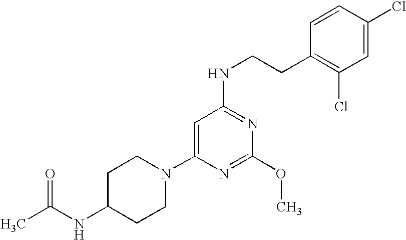

To a mixture of [6-(3-amino-piperidin-1-yl)-2-methoxy-pyrimidin-4-yl]-[2-(2,4-dichloro-phenyl)-ethyl]-amine (190 mg), triethylamine (134 μL, 0.96 mmol), and N,N-dimethylaminopyridine (6 mg) in tetrahydrofuran (6 mL) is added acetyl chloride (41 μL, 0.58 mmol). The reaction mixture is stirred for 17 hours, quenched with the addition of water (20 mL) and extracted twice with ethyl acetate (25 mL). The organic extracts are combined, dried over magnesium sulfate, concentrated, and purified via silica gel chromatography (12 g) eluting with 0 to 12% MeOH in CH₂Cl₂ to give N-(1-{6-[2-(2,4-dichloro-phenyl)-ethylamino]-2-methoxy-pyrimidin-4-yl}-1-piperidin-4-yl)-acetamide (48 mg) as a solid. LCMS R_{T}=1.9 minutes, MS: 438 (M+H). ¹H NMR [300 MHz, (CD₃)₂SO]: δ 7.81 (1H, d); 7.59 (1H, s); 7.36 (2H, s); 6.79 (2H, m); 5.31 (1H, s); 4.07 (2H, m); 3.78 (1H, d); 3.71 (3H, s); 3.41 (2H, m); 2.91 (4H, m); 1.78 (3H, s); 1.73 (1H, m); 1.25 (4H m). IC₅₀=26 nM.

### Example 6

### 5-{6-[2-(2,4-Dichloro-phenyl)-ethylamino]-2-methoxy-pyrimidin-4-yl}-1-methyl-2,3-dihydro-1H-indole-2-carboxylic acid

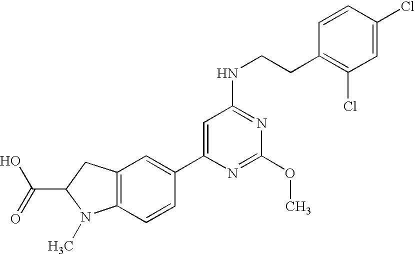

Step 1. To a mixture of ethyl-5-bromoindole-2-carboxylate (2.5 g) in DMF (20 mL) is added a solution of 60% NaH (485 mg) in DMF (10 mL). The resulting mixture is stirred for 15 minutes and iodomethane (0.638 mL) is added by syringe. The reaction mixture is allowed to stir at ambient temperature for 20 hours. Water is added (200 mL) and the mixture is extracted twice with ethyl acetate (100 mL). The organic extracts are combined, washed with water (3×50 mL) and once with brine (50 mL), and dried over magnesium sulfate, and concentrated to give 5-bromo-1-methyl-1H-indole-2-carboxylic acid ethyl ester (1.28 g) as a solid. ¹H NMR [300 MHz, CDCl₃]: δ 7.79 (1H, d); 7.41 (1H, dd); 7.27 (1H, t); 7.2 (1H, s); 4.39 (2H, q); 4.05 (3H, s); 1.41 (3H, t).

Step 2. To a solution of 5-bromo-1-methyl-1H-indole-2-carboxylic acid ethyl ester (1.28 g) in trifluoroacetic acid (10 mL), is added sodiumcyanoborohydride (680 mg) at 0° C. The reaction mixture is allowed to warm up to ambient temperature, stirred for 20 hours and quenched with water (100 mL). The mixture is made basic with NaOH, and extracted with Et₂O (3×50 mL). The organic extracts are combined, washed with brine (30 mL), dried over magnesium sulfate, concentrated, and purified via silica gel chromatography (34 g) eluting with 0 to 25% ethyl acetate in heptane to give 5-bromo-1-methyl-2,3-dihydro-1H-indole-2-carboxylic acid ethyl ester (800 mg) as a solid. ¹H NMR [300 MHz, CDCl₃]: δ 7.19 (1H, d); 7.21 (1H, s); 6.34 (1H, d); 4.25 (2H, qd); 4.06 (1H, t); 3.21 (2H, m); 2.82 (3H, s); 1.30 (3H, t).

Step 3. A mixture of 5-bromo-1-methyl-2,3-dihydro-1H-indole-2-carboxylic acid ethyl ester (800 mg), bis(pinacolato)diboron (1.5 g), potassium acetate (1.47 g), and PdCl₂(dppf)₂ (139 mg) in dimethylsulfoxide (10 mL) is degassed with bubbling nitrogen for 5 minutes. The mixture is heated to 90° C. for 4 hours. The reaction mixture is cooled, diluted with water (75 mL) and ethyl acetate (100 mL), and stirred in decolorizing carbon. The biphasic mixture is filtered through celite and the filtrate is extracted twice with EtOAc (50 mL). The organic extracts are combined, washed thrice with water (50 mL), once with brine (30 mL), dried over magnesium sulfate, concentrated, and purified via silica gel chromatography (34 g) eluting with 0 to 20% ethyl acetate in heptane to give 1-methyl-5-(4,4,5,5-tetramethyl-[1,3,2]dioxaborolan-2-yl)-2,3-dihydro-1H-indole-2-carboxylic acid ethyl ester (903 mg) as a solid. ¹H NMR [300 MHz, (CD₃)₂SO]: δ 7.39 (1H, d); 7.28 (1H, s); 6.46 (1H, d); 4.18 (3H, m); 3.3 (1H, d); 2.97 (1H, m); 2.79 (3H, s); 1.24 (12H, s); 1.22 (3H, t).

Step 4: A mixture of (6-chloro-2-methoxy-pyrimidin-4-yl)-[2-(2,4-dichloro-phenyl)-ethyl]-amine (200 mg), 1-methyl-5-(4,4,5,5-tetramethyl-[1,3,2]dioxaborolan-2-yl)-2,3-dihydro-1H-indole-2-carboxylic acid ethyl ester (300 mg), Cs₂CO₃ (390 mg) and tetrakis(triphenylphosphine) palladium (35 mg) in water (0.4 mL) and ethylene glycol dimethyl ether (1.6 mL) is degassed with bubbling nitrogen for 5 minutes and heated at 90° C. for 19 hours. The reaction mixture is cooled, diluted with water (50 mL) and extracted twice with ethyl acetate (50 mL). The organic extracts are combined, dried over magnesium sulfate, concentrated, and purified via silica gel chromatography (40 g) eluting with 0 to 40% ethyl acetate in heptane to give 5-{6-[2-(2,4-dichloro-phenyl)-ethylamino]-2-methoxy-pyrimidin-4-yl}-1-methyl-2,3-dihydro-1H-indole-2-carboxylic acid ethyl ester (110 mg) as a solid. LCMS R_{T}=5.57 minutes, MS: 501 (M+H). ¹H NMR [300 MHz, (CD₃)₂SO]: δ 7.72 (2H, m); 7.59 (1H, s); 7.37 (3H, s); 6.54 (1H, d); 6.42 (1H, s); 4.30 (1H, m); 4.17 (2H, qd); 3.84 (3H, s); 3.54 (2H, b); 3.41 (1H, m); 3.06 (1H, m); 2.97 (2H, t); 2.83 (3H, s); 1.23 (3H, t).

Step 5: Lithium hydroxide monohydrate (1.28 mmol) is added to a stirred solution of 5-{6-[2-(2,4-dichloro-phenyl)-ethylamino]-2-methoxy-pyrimidin-4-yl}-1-methyl-2,3-dihydro-1H-indole-2-carboxylic acid ethyl ester (0.43 mmol) in MeOH/H₂O (10 mL, 9:1). The reaction mixture is stirred overnight at room temperature. The reaction is diluted with water and volatiles are removed in vacuo. The aqueous residue is extracted once with Et₂O, acidified (1N, HCl) to pH 4, and extracted twice with ethyl acetate. The combined organic layer is dried (MgSO₄) and concentrated in vacuo to afford 5-{6-[2-(2,4-dichloro-phenyl)-ethylamino]-2-methoxy-pyrimidin-4-yl}-1-methyl-2,3-dihydro-1H-indole-2-carboxylic acid.

### Example 7

### (a) 2-Methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide

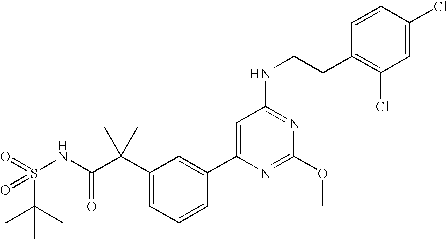

Step 1: To a solution of LDA in THF/n-heptane/ethylbenzene (1.8 M, 17 mL) at 0° C. is added a solution of 2-(3-bromo-phenyl)-propionic acid (3 g) in THF (5 mL) dropwise during 15 minutes. The mixture is stirred for 1 hour followed by addition of methyl iodide (4.93 g) in THF (5 mL) dropwise during 10 min. The reaction mixture is stirred for 15 hours, quenched with 2N hydrochloric acid, concentrated in vacuo, and diluted with ether (150 mL). The ether layer is washed with 2N hydrochloric acid, and extracted three times with 2N sodium hydroxide (50 mL). The combined sodium hydroxide layers are acidified with 6 N hydrochloric acid to pH 1 and extracted three times with ether (75 mL). The combined organic layers are washed with brine, dried over sodium sulfate and concentrated to obtain 2-(3-bromo-phenyl)-2-methyl-propionic acid as a solid (3.08 g), which is used without further purification. LC/MS: 243 (M+H)

Step 2: A solution of 2-(3-bromo-phenyl)-2-methyl-propionic acid (2.18 mmol) in anhydrous ether (20 mL) is added tert-butyl lithium (1.7 M in pentane, 5.4 mL, 9.16 mmol) dropwise at −78° C. and this mixture is stirred for 30 minutes treated with tributyl borate (2.34 mL, 8.72 mmol). The reaction mixture is allowed to warm up to room temperature, stirred for 15 hours, diluted with ether, and quenched with 1 M H₃PO₄. After stirring for 30 minutes the ether layer is separated and extracted with 2 N aqueous sodium hydroxide (3×20 mL). The combined sodium hydroxide extracts are acidified with 6 N hydrochloric acid to pH 1 and extracted three times with ether (50 mL). The combined organic extracts are washed with brine, dried over sodium sulfate and concentrated to obtain 3-(1-carboxy-1-methyl-ethyl)-phenyl boronic acid, which is used without further purification.

Step 3: A solution of (6-chloro-2-methoxy-pyrimidin-4-yl)-[2-(2,4-dichloro-phenyl)-ethyl]-amine (0.51 mmol) and 3-(1-carboxy-1-methyl-ethyl)-phenyl boronic acid (0.61 mmol) in MeCN (2.5 mL) and aqueous Na₂CO₃ solution (0.4 M, 2.5 mL) is degassed with nitrogen for 5 minutes before addition of tetrakis(triphenylphosphine) palladium (0) (29.5 mg). The reaction vessel is sealed and heated under microwave to 130° C. for 30 minutes. To the reaction mixture is added 2 mL of water, the pH is adjusted to 7 using 2 N aqueous hydrochloric acid and this mixture is extracted three times with EtOAc (30 mL). The combined extracts are washed with brine, dried over sodium sulfate and concentrated. The resulting oil is subjected to silica gel chromatography eluting with 0 to 7% MeOH in DCM to give 2-(3-{6-[2-(2,4-dichloro-phenyl)-ethylamino]-2-methoxy-pyrimidin-4-yl}-phenyl)-2-methyl-propionic acid (205 mg) as a solid. LC/MS: R_{T}=2.39 minutes, MS: 460.2 (M+H). ¹H NMR [300 MHz, (CD₃)₂SO]: δ 12.38 (1H, s), 7.36-8 (7H, m), 6.58 (1H, s), 3.84 (3H, s), 3.58 (2H, m), 2.98 (2H, m), 1.54 (6H, s).

Step 4: N-(3-dimethylaminopropyl)-N-ethylcarbodiimide hydrochloride (0.23 mmol) is added to a stirred ice cold solution of 2-(3-{6-[2-(2,4-dichloro-phenyl)-ethylamino]-2-methoxy-pyrimidin-4-yl}-phenyl)-2-methyl-propionic acid (0.22 mmol), tert-butylsulfonamide (0.23 mmol) and 4-dimethylaminopyridine (0.22 mmol) in dry DCM under nitrogen atmosphere. The ice bath is removed and the reaction mixture is stirred overnight at 60° C. The volatiles are removed under reduced pressure, the residue is dissolved in ethyl acetate, washed with 0.1 N HCl, brine and water, dried over sodium sulfate, filtered and concentrated under reduced pressure. The crude residue is purified by chromatography (SiO₂ packed column), eluting with EtOAc/DCM to afford 2-methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide (25 mg). LCMS: R_{T}=2.67 minutes, MS: 579, 581 (M+H). IC₅₀=2 nM.

### (b) N,N-dimethylamide-2-sulfonic acid [2-(3-{6-[2-(2,4-dichloro-phenyl)-ethylamino]-2-methoxy-pyrimidin-4-yl}-phenyl)-2-methyl-propionyl]-amide

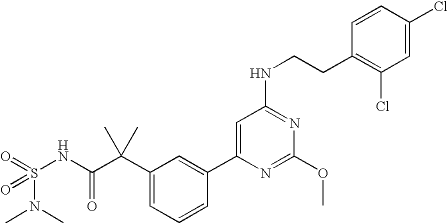

By proceeding in a similar manner as Example 7(a), step 4, but substituting N,N-dimethylsulfamide for tert-butylsulfonamide, there is prepared N,N-dimethylamide-2-sulfonic acid [2-(3-{6-[2-(2,4-dichloro-phenyl)-ethylamino]-2-methoxy-pyrimidin-4-yl}-phenyl)-2-methyl-propionyl]-amide (185 mg). LCMS: R_{T}=2.26 minutes, MS: 566, 568 (M+H).

### (c) 2-(3-{6-[2-(2,4-Dichloro-phenyl)-ethylamino]-2-methoxy-pyrimidin-4-yl}-phenyl)-2-methyl-1-thiomorpholin-4-yl-propan-1-one

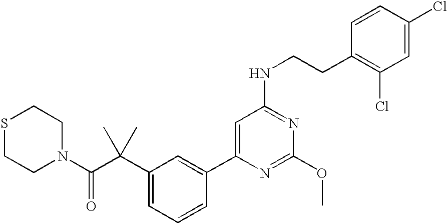

By proceeding in a similar manner as Example 7(a), step 4, but substituting thiomorpholine for tert-butylsulfonamide, there is prepared 2-(3-{6-[2-(2,4-dichloro-phenyl)-ethylamino]-2-methoxy-pyrimidin-4-yl}-phenyl)-2-methyl-1-thiomorpholin-4-yl-propan-1-one (120 mg). LCMS: R_{T}=2.68 minutes, MS: 545, 547 (M+H). IC₅₀=383 nM

### (d) 2-(3-{6-[2-(2,4-Dichloro-phenyl)-ethylamino]-2-methoxy-pyrimidin-4-yl}-phenyl)-isobutyramide

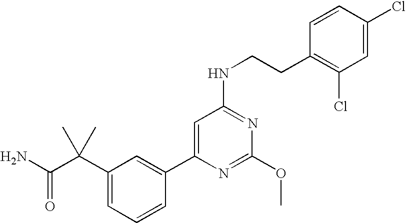

By proceeding in a similar manner as Example 7(a), step 4, but substituting ammonium bicarbonate for tert-butylsulfonamide, there is prepared 2-(3-{6-[2-(2,4-dichloro-phenyl)-ethylamino]-2-methoxy-pyrimidin-4-yl}-phenyl)-isobutyramide (120 mg). LCMS: R_{T}=2.01 minutes, MS: 459, 461 (M+H).

### (e) 2-(3-{6-[2-(2,4-Dichloro-phenyl)-ethylamino]-2-methoxy-pyrimidin-4-yl}-phenyl)-N,N-dimethyl-isobutyramide

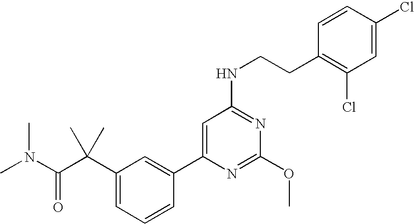

By proceeding in a similar manner as on Example 7(a), step 4, but substituting dimethylamine for tert-butylsulfonamide, there is prepared 2-(3-{6-[2-(2,4-dichloro-phenyl)-ethylamino]-2-methoxy-pyrimidin-4-yl}-phenyl)-N,N-dimethyl-isobutyramide (186 mg). LCMS: R_{T}=2.44 minutes, MS: 487, 489 (M+H).

### Example 8

### (1-{6-[2-(2,4-Dichloro-phenyl)-ethylamino]-2-methoxy-pyrimidin-4-yl}-piperidin-3-yl)-acetic acid

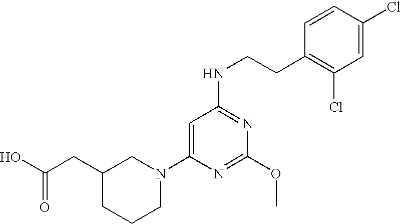

Step 1: A solution of (6-chloro-2-methoxy-pyrimidin-4-yl)-[2-(2,4-dichloro-phenyl)-ethyl]-amine (3 mmol), piperidine acetic acid ethyl ester (7.5 mmol) and K₂CO₃ (9 mmol) in 1-methyl-2-pyrrolidinone (10 mL) is stirred overnight at 145° C. The reaction is cooled to room temperature, diluted with water (60 mL) and extracted twice with DCM. The aqueous layer is acidified slowly with 1N hydrochloric acid to pH 4 while stirring vigorously, and the stirring is continued for 1.5 hours. The formed precipitate is suction filtered and air dried to afford (1-{6-[2-(2,4-dichloro-phenyl)-ethylamino]-2-methoxy-pyrimidin-4-yl}-piperidin-3-yl)-acetic acid ethyl ester (1.42 g). LCMS: R_{T}=2.35 minutes, MS: 467, 469 (M+H).

Step 2: Lithium hydroxide monohydrate (54 mg) is added to a stirred solution of (1-{6-[2-(2,4-dichloro-phenyl)-ethylamino]-2-methoxy-pyrimidin-4-yl}-piperidin-3-yl)-acetic acid ethyl ester (0.2 g) in MeOH/H₂O (10 mL, 9:1). The reaction mixture is stirred overnight at room temperature. The reaction is diluted with water and volatiles are removed in vacuo. The aqueous residue is extracted once with Et₂O, acidified (1N, HCl) to pH 4, and extracted twice with ethyl acetate. The combined organic layer is dried (MgSO₄) and concentrated in vacuo to afford (1-{6-[2-(2,4-dichloro-phenyl)-ethylamino]-2-methoxy-pyrimidin-4-yl}-piperidin-3-yl)-acetic acid (180 mg). LCMS: R_{T}=2.08 minutes, MS: 439, 441 (M+H). IC₅₀=0.5 nM.

### Example 9

### 1-{2-Methoxy-6-[2-(4-trifluoromethoxy-phenyl)-ethylamino]-pyrimidin-4-yl}-piperidine-3-carboxylic acid

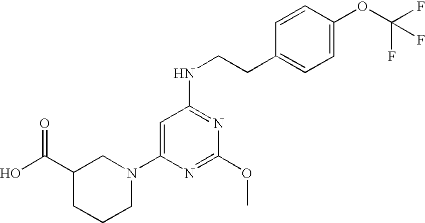

A solution of (6-chloro-2-methoxy-pyrimidin-4-yl)-[2-(4-trifluoromethoxy-phenyl)-ethyl]-amine (1 g), nipecotic acid (0.93 g) and K₂CO₃ (1.19 g) in 1-methyl-2-pyrrolidinone (10 mL) is stirred overnight at 145° C. The reaction is cooled to room temperature, diluted with water (60 mL) and extracted twice with dichloromethane. The aqueous layer is acidified slowly with 1N hydrochloric acid to pH 4 while stirring vigorously, and the stirring is continued for 1.5 hours. The formed precipitate is suction filtered and air dried to afford 1-{2-methoxy-6-[2-(4-trifluoromethoxy-phenyl)-ethylamino]-pyrimidin-4-yl}-piperidine-3-carboxylic acid as a powder (0.99 g). LCMS: R_{T}=2.07 minutes, MS: 441 (M+H). IC₅₀=9 nM

### Example 10

### N-(1-{2-Methoxy-6-[2-(4-trifluoromethoxy-phenyl)-ethylamino]-pyrimidin-4-yl}-piperidine-3-carbonyl)-methanesulfonamide

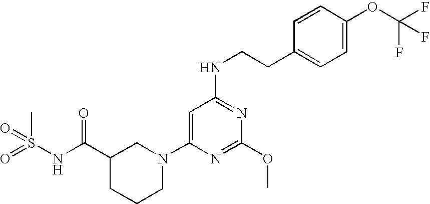

N-(3-dimethylaminopropyl)-N-ethylcarbodiimide hydrochloride (68 mg) is added to a stirred ice cold solution of 1-{2-methoxy-6-[2-(4-trifluoromethoxy-phenyl)-ethylamino]-pyrimidin-4-yl}-piperidine-3-carboxylic acid (150 mg), methanesulfonamide (48.6 mg) and 4-dimethylaminopyridine (50 mg) in dry DCM under N₂. The ice bath is removed and the reaction mixture is stirred overnight, while warming to room temperature. The mixture is concentrated in vacuo. The residue is dissolved in ethyl acetate, washed with 0.1N HCl, brine and water, dried (Na₂SO₄), filtered and concentrated. The residue is purified by chromatography (SiO₂ packed column), eluted with EtOAc/DCM to afford N-(1-{2-Methoxy-6-[2-(4-trifluoromethoxy-phenyl)-ethylamino]-pyrimidin-4-yl}-piperidine-3-carbonyl)-methanesulfonamide (65 mg). LCMS: R_{T}=2.09 minutes, MS: 518 (M+H). IC₅₀=22 nM.

### Example 11

### (a) N-(1-{6-[2-(2,4-Dichloro-phenyl)-ethylamino]-2-methoxy-pyrimidin-4-yl}-piperidine-3-carbonyl)-methanesulfonamide

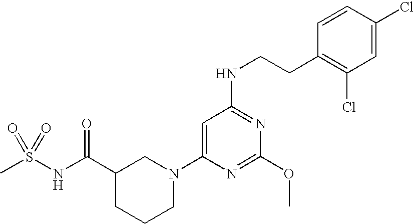

Step 1: In a tube is combined (6-chloro-2-methoxy-pyrimidin-4-yl)-[2-(2,4-dichloro-phenyl)-ethyl]-amine (200 mg), nipecotic acid (194 mg), K₂CO₃ (249 mg) and 1-methyl-2-pyrrolidinone (2.5 mL). The tube is sealed and heated to 140° C. and stirred for 5 hours. The mixture is allowed to cool to ambient temperature, stand for 12 hours, diluted with water (20 mL) and acidified using 3M aqueous HCl. A precipitate forms and is collected by filtration and dried under high vacuum to afford 1-{6-[2-(2,4-dichloro-phenyl)-ethylamino]-2-methoxy-pyrimidin-4-yl}-piperidine-3-carboxylic acid (121 mg) as a solid. LCMS R_{T}=2.15 minutes, MS: 425 (M+H).

Step 2: N-(3-dimethylaminopropyl)-N-ethylcarbodiimide hydrochloride (71 mg) is added to a stirred ice cold solution of 1-{6-[2-(2,4-dichloro-phenyl)-ethylamino]-2-methoxy-pyrimidin-4-yl}-piperidine-3-carboxylic acid (150 mg), methanesulfonamide (43.6 mg) and 4-dimethylaminopyridine (52 mg) in dry dichloromethane under N₂. The ice bath is removed and the reaction mixture is stirred overnight at room temperature. The mixture is concentrated in vacuo. The residue is dissolved in ethyl acetate, washed with 0.1N HCl, brine and water, dried (Na₂SO₄), filtered and concentrated. The crude is purified by chromatography (SiO₂ packed column), eluting with EtOAc/DCM to give N-(1-{6-[2-(2,4-dichloro-phenyl)-ethylamino]-2-methoxy-pyrimidin-4-yl}-piperidine-3-carbonyl)-methanesulfonamide (145 mg). LCMS: R_{T}=1.88 minutes, MS: 502, 504 (M+H). IC₅₀=1 nM.

### (b) Ethanesulfonic acid (1-{6-[2-(2,4-dichloro-phenyl)-ethylamino]-2-methoxy-pyrimidin-4-yl}-piperidine-3-carbonyl)-amide

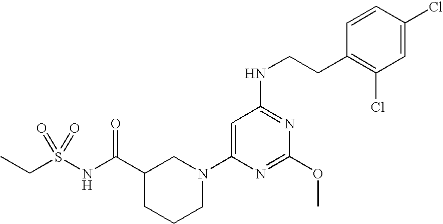

By proceeding in a similar manner as Example 11(a), step 2, but substituting ethanesulfonamide for methanesulfonamide, there is prepared ethanesulfonic acid (1-{6-[2-(2,4-dichloro-phenyl)-ethylamino]-2-methoxy-pyrimidin-4-yl}-piperidine-3-carbonyl)-amide (125 mg). LCMS: R_{T}=2.12 minutes, MS: 516, 518 (M+H).

### (c) 2-Methyl-propane-2-sulfonic acid (1-{6-[2-(2,4-dichloro-phenyl)-ethylamino]-2-methoxy-pyrimidin-4-yl}-piperidine-3-carbonyl)-amide

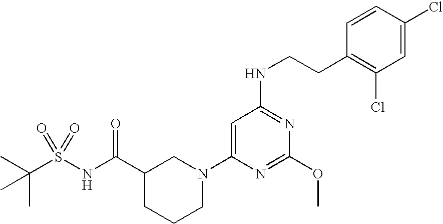

By proceeding in a similar manner as Example 11(a), step 2, but substituting tert-butylsulfonamide for methanesulfonamide, there is prepared 2-methyl-propane-2-sulfonic acid (1-{6-[2-(2,4-dichloro-phenyl)-ethylamino]-2-methoxy-pyrimidin-4-yl}-piperidine-3-carbonyl)-amide (132 mg). LCMS: R_{T}=2.2 minutes, MS: 544, 546 (M+H).

### (d) N-(1-{6-[2-(2,4-Dichloro-phenyl)-ethylamino]-2-methoxy-pyrimidin-4-yl}-piperidine-3-carbonyl)-C,C,C-trifluoro-methanesulfonamide

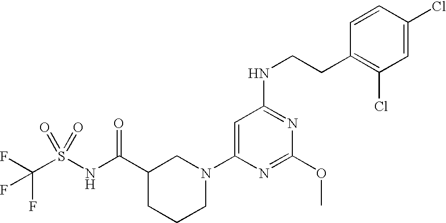

By proceeding in a similar manner as Example 11(a), step 2, but substituting trifluoromethyl sulfonamide for methanesulfonamide, there is prepared N-(1-{6-[2-(2,4-dichloro-phenyl)-ethylamino]-2-methoxy-pyrimidin-4-yl}-piperidine-3-carbonyl)-C,C,C-trifluoro-methanesulfonamide (257 mg). LCMS: R_{T}=2.3 minutes, MS: 556, 558 (M+H). IC₅₀=18 nM.

### (e) 1-{6-[2-(2,4-Dichloro-phenyl)-ethylamino]-2-methoxy-pyrimidin-4-yl}-piperidine-3-carboxylic acid (1H-tetrazol-5-yl)-amide

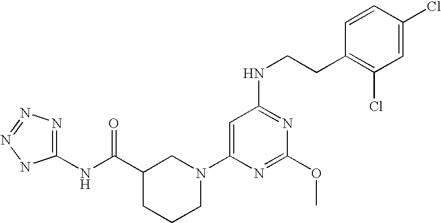

By proceeding in a similar manner as Example 11(a), step 2, but substituting 1H-tetrazol-5-ylamine for methanesulfonamide, there is prepared 1-{6-[2-(2,4-dichloro-phenyl)-ethylamino]-2-methoxy-pyrimidin-4-yl}-piperidine-3-carboxylic acid (1H-tetrazol-5-yl)-amide (15 mg). LCMS: R_{T}=1.81 minutes, MS: 492, 494 (M+H). IC₅₀=4.4 nM.

### (f) 1-{6-[2-(2,4-Dichloro-phenyl)-ethylamino]-2-methoxy-pyrimidin-4-yl}-piperidine-3-carboxylic acid amide

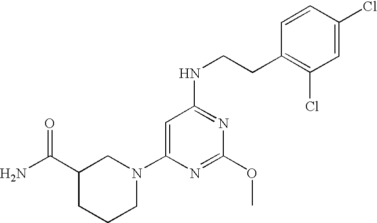

N-(3-dimethylaminopropyl)-N-ethylcarbodiimide hydrochloride (0.23 mmol) is added to a stirred ice cold solution of 1-{6-[2-(2,4-dichloro-phenyl)-ethylamino]-2-methoxy-pyrimidin-4-yl}-piperidine-3-carboxylic acid (0.22 mmol), ethanesulfonamide (0.23 mmol) and 4-dimethylaminopyridine (0.22 mmol) in dry dichloromethane under N₂. The ice bath is removed and the reaction mixture is stirred overnight at 60° C. The mixture is concentrated in vacuo. The residue is dissolved in ethyl acetate, washed with 0.1N HCl, brine and water, dried (Na₂SO₄), filtered and concentrated under reduced pressure. The crude residue was purified by chromatography (SiO₂ packed column), eluting with EtOAc/DCM to give 1-{6-[2-(2,4-dichloro-phenyl)-ethylamino]-2-methoxy-pyrimidin-4-yl}-piperidine-3-carboxylic acid amide (75 mgs). LCMS: R_{T}=1.77 minutes, MS: 424, 426 (M+H).

### (g) 1-{6-[2-(2,4-Dichloro-phenyl)-ethylamino]-2-methoxy-pyrimidin-4-yl}-piperidine-3-carboxylic acid dimethylamide

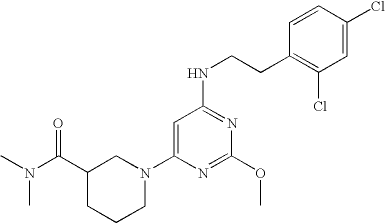

By proceeding in a similar manner as Example 11(a), step 2, but substituting dimethylamine for methanesulfonamide, there is prepared 1-{6-[2-(2,4-dichloro-phenyl)-ethylamino]-2-methoxy-pyrimidin-4-yl}-piperidine-3-carboxylic acid dimethylamide (65 mg). LCMS: R_{T}=1.88 minutes, MS: 452, 454 (M+H).

### (h) N,N-Dimethylamide-2-sulfonic acid 1-{6-[2-(2,4-dichloro-phenyl)-ethylamino]-2-methoxy-pyrimidin-4-yl}-piperidine-3-carboxamide

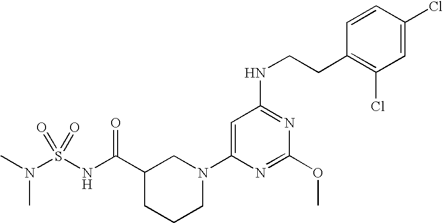

By proceeding in a similar manner as Example 11(a), step 2, but substituting N,N-dimethylsulfamide for methanesulfonamide, there is prepared N,N-dimethylamide-2-sulfonic acid 1-{6-[2-(2,4-dichloro-phenyl)-ethylamino]-2-methoxy-pyrimidin-4-yl}-piperidine-3-carboxamide (241 mgs). LCMS: R_{T}=2.5 minutes, MS: 531, 533 (M+H). IC₅₀=14 nM.

### Example 12

### 5-{2-Methoxy-6-[2-(4-trifluoromethoxy-phenyl)-ethylamino]-pyrimidin-4-yl}-thiophene-2-carboxylic acid

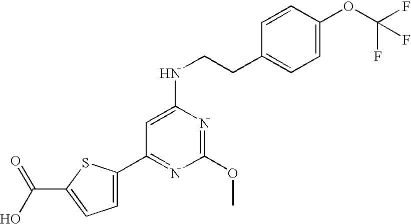

Step 1: 5-(Dihydroxyboryl)-2-thiophenecarboxylic acid (527 mg) and 2,2-dimethyl-propane-1,3-diol (361 mg) are stirred at room temperature in THF (10 mL) for 19 hours and concentrated in vacuo to afford 5-(5,5-dimethyl-[1,3,2]dioxaborinan-2-yl)-thiophene-2-carboxylic acid (748 mg) as a solid. LCMS: R_{T}=1.15 minutes; ¹H NMR [300 MHz, (CD₃)₂SO]: □ 13.15 (1H, s); 7.7 (1H, m); 7.45 (1H, m); 3.75 (4H, s); 0.95 (6H, s).

Step 2: A mixture of (6-chloro-2-methoxy-pyrimidin-4-yl)-[2-(4-trifluoromethoxy-phenyl)-ethyl]-amine (267 mg), 5-(5,5-dimethyl-[1,3,2]dioxaborinan-2-yl)-thiophene-2-carboxylic acid (277 mg), cesium fluoride (351 mg), and tetrakis(triphenylphosphine) palladium (71 mg) in water (1.6 mL) and ethylene glycol dimethyl ether (6.4 mL) is degassed with bubbling nitrogen for 5 minutes and is heated to 85° C. for 16 hours. The reaction mixture is cooled, diluted with water (150 mL) and brine (25 mL), and extracted two times with EtOAc (100 mL) and the extracts are concentrated in vacuo. The residue is subjected to flash column chromatography on silica (10 g) eluting with 0 to 5% MeOH in EtOAc. The resulting crystalline solid is triturated with DCM (5 mL) and ether (5 mL) and dried to afford 5-{2-methoxy-6-[2-(4-trifluoromethoxy-phenyl)-ethylamino]-pyrimidin-4-yl}-thiophene-2-carboxylic acid (42 mg) as a solid. MS: 440; LCMS: R_{T}=3.48 minutes; ¹H NMR [300 MHz, (CD₃)₂SO]: □ 7.7 (3H, m); 7.35 (2H, m); 7.25 (2H, m), 6.6 (1H, s); 3.85 (3H, s); 2.55 (2H; m); 1.9 (2H, t, J=7 Hz). IC₅₀=2 nM.

### Example 13

### 5-{6-[2-(2,4-Dichloro-phenyl)-ethylamino]-2-methoxy-pyrimidin-4-yl}-2,3-dihydro-benzofuran-2-carboxylic acid hydrochloride

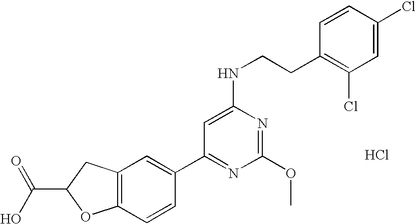

Step 1: To a solution of 2,3-dihydro-benzofuran-2-carboxylic acid (510 mg) in glacial acetic acid (4 mL) is added bromine (497 mg) dropwise. After 16 hours, the reaction is quenched with water (100 mL) and sodium bisulfite (1 g) and extracted twice with EtOAc (100 mL). The extracts are concentrated in vacuo and dried under high vacuum to afford 5-bromo-2,3-dihydro-benzofuran-2-carboxylic acid (811 mg) as a solid. MS: 241 (M+H), ¹H NMR [300 MHz, (CD₃)₂SO]: δ 13.05 (1H, s); 7.4 (1H, s); 7.25 (1H, d); 6.8 (1H, m); 5.25 (1H, q), 3.55 (1H, dd); 3.25 (1H, m).

Step 2: A mixture of 5-bromo-2,3-dihydro-benzofuran-2-carboxylic acid (0.74 g), bis(pinacolato)diboron (1.51 g), potassium acetate (1.47 g, 15 mmol), and PdCl₂(dppf)₂ (115 mg, 0.14 mmol) in dimethylsulfoxide (10 mL) is degassed with bubbling nitrogen for 5 minutes. The mixture is heated to 90° C. for 16 hours. The reaction mixture is cooled, diluted with water (200 mL) and brine (25 mL), and filtered through Celite followed by water (200 mL) and EtOAc (200 mL). The filtrate is extracted twice with EtOAc (200 mL) and the extracts are concentrated in vacuo. The residue is subjected to flash column chromatography on silica (4 g) eluting with 80 to 100% EtOAc in heptane to afford 5-(4,4,5,5-tetramethyl-[1,3,2]dioxaborolan-2-yl)-2,3-dihydro-benzofuran-2-carboxylic acid (715 mg) as an oil. MS: 289 (M−H), ¹H NMR [300 MHz, (CD₃)₂SO]: δ 13.05 (1H, s); 7.5 (2H, m); 6.8 (1H, m); 5.2 (1H, m); 3.6 (1H, m); 3.3 (1H, m); 1.05 (12H, s).

Step 3: A mixture of (6-chloro-2-methoxy-pyrimidin-4-yl)-[2-(2,4-dichloro-phenyl)-ethyl]-amine (212 mg), 5-(4,4,5,5-tetramethyl-[1,3,2]dioxaborolan-2-yl)-2,3-dihydro-benzofuran-2-carboxylic acid (124 mg), cesium carbonate (414 mg), and tetrakis(triphenylphosphine) palladium (49 mg) in water (1.2 mL) and ethylene glycol dimethyl ether (4.8 mL) is degassed with bubbling nitrogen for 5 minutes and is heated to 70° C. for 64 hours. The reaction mixture is cooled, diluted with water (150 mL) and brine (25 mL), and extracted two times with EtOAc (150 mL) and the extracts are concentrated in vacuo. The residue is subjected to flash column chromatography on silica (4 g) eluting with 0 to 25% MeOH in EtOAc to afford 5-{6-[2-(2,4-dichloro-phenyl)-ethylamino]-2-methoxy-pyrimidin-4-yl}-2,3-dihydro-benzofuran-2-carboxylic acid (80 mg) as an oil. MS: 460; LCMS: R_{T}=2.81 minutes.

Step 4: A portion of 5-{6-[2-(2,4-dichloro-phenyl)-ethylamino]-2-methoxy-pyrimidin-4-yl}-2,3-dihydro-benzofuran-2-carboxylic acid is subjected to flash column chromatography on silica (5 g) eluting with 0 to 25% MeOH in EtOAc. The product is dissolved in MeOH and treated with 0.5 M hydrogen chloride in MeOH and concentrated in vacuo. The product is dissolved in THF (3 mL) and ether (10 mL) is added. The precipitate is removed and dried to afford 5-{6-[2-(2,4-dichloro-phenyl)-ethylamino]-2-methoxy-pyrimidin-4-yl}-2,3-dihydro-benzofuran-2-carboxylic acid hydrochloride (20 mg) as a solid. LCMS: R_{T}=2.79 minutes; MS: 460. IC₅₀=2 nM.

### Pharmacological Testing

The inhibitory effects of the compounds according to the invention are assessed in a human DP functional assay. A cAMP assay is employed using the human cell line LS174T, which expresses the endogenous DP receptor. The protocol is similar to that described previously (Wright D H, Ford-Hutchinson A W, Chadee K, Metters K M, The human prostanoid DP receptor stimulates mucin secretion in LS174T cells, Br J Pharmacol. 131(8):1537-45 (2000)).

Protocol for SPA cAMP Assay in Human LS174 T Cells

### Materials

PGD2 (Cayman Chemical Cat #12010)
IBMX (Sigma Cat #5879)
cAMP SPA direct screening assay system (Amersham code RPA 559)
96-well cell plates (Wallac Cat #1450-516)
Wallac 1450 Microplate Trilux scintillation counter (PerkinElmer)
Plate sealers
Eppendorf tubes
Dulbecco's Phosphate-Buffered Saline (PBS) (Invitrogen Cat #14040-133)
Distilled water
Vortex
Magnetic stirrer and stirrer bars

### Reagent Preparation:

All reagents should be allowed to equilibrate to room temperature before reconstitution.

### 1× Assay Buffer

Transfer the contents of the bottle to a 500 mL graduated cylinder by repeated washing with distilled water. Adjust the final volume to 500 mL with distilled water and mix thoroughly.

### Lysis Reagent 1 & 2

Dissolve each of the lysis reagents 1 and 2 in 200 mL assay buffer respectively. Leave at room temperature for 20 minutes to dissolve.

### SPA Anti-Rabbit Beads

Add 30 mL of lysis buffer 2 to the bottle. Gently shake the bottle for 5 minutes.

### Antiserum

Add 15 mL of lysis buffer 2 to each vial, and gently mix until the contents are completely dissolved.

Tracer (I¹²⁵-cAMP)

Add 14 mL lysis buffer 2 to each vial and gently mix until the contents are completely dissolved.

### Preparation of Immunoreagent

1) Add equal volumes of tracer, antiserum and SPA anti-rabbit reagent to a bottle, ensuring that a sufficient volume of this mixture is prepared for the desired number of wells (150 μL/well).
2) Mix thoroughly.
3) This immunoreagent solution should be freshly prepared before each assay and not re-used.

### Standard

1) Add 1 mL lysis buffer 1 and gently mix until contents are completely dissolved.
2) The final solution contains cAMP at a concentration of 512 pmol/mL.
3) Label 7 polypropylene or polystyrene tubes, 0.2 pmol, 0.4 pmol, 0.8 pmol, 1.6 pmol, 3.2 pmol, 6.4 pmol and 12.8 pmol.
4) Pipette 500 μL of lysis buffer 1 into all the tubes.
5) Into the 12.8 pmol tube pipette 500 μL of stock standard (512 pmol/mL) and mix thoroughly. Transfer 500 μL from 12.8 pmol tube to the 6.4 pmol tube and mix thoroughly. Repeat this doubling dilution successively with the remaining tubes.
6) 50 μL aliquots in duplicate from each serial dilution and the stock standard will give rise to 8 standard levels of cAMP ranging from 0.2-25.6 pmol standard

### Compound Dilution Buffer

Add 50 μL of 1 mM IBMX into 100 mL PBS to make a final concentration of 100 μM and sonicate at 30° C. for 20 minutes.

### PGD2 Preparation

Dissolve 1 mg PGD2 (FW, 352.5) in 284 μL DMSO to make 10 mM stock solution and store at 20° C. Before each assay, it is freshly prepared. Add 3 μL of 10 mM stock solution to 20 mL DMSO, mix it thoroughly, and transfer 10 mL to 40 mL PBS.

### Compound Dilution

Compound dilution is carried out in Biomex 2000 (Beckman) using Method 1_cAMP DP 11 points.

5 μL of each compound from the 10 mM stock compound plates is transferred to the wells of a 96-well plate respectively as below.

| | 1 | 2 | 3 | 4 | 5 | 6 | 7 | 8 | 9 | 10 | 11 | 12 |
|---|---|---|---|---|---|---|---|---|---|---|---|---|
| A | 1 | | | | | | | | | | | |
| B | 2 | | | | | | | | | | | |
| C | 3 | | | | | | | | | | | |
| D | 4 | | | | | | | | | | | |
| E | 5 | | | | | | | | | | | |
| F | 6 | | | | | | | | | | | |
| G | 7 | | | | | | | | | | | |
| H | reference | | | | | | | | | | | |

Fill the plate with 45 μL of DMSO except column 7 is filled with 28 μL DMSO. Pipette column 1 thoroughly, and transfer 12 μL into column 7 parallel. Perform 1:10 serial dilution from column 1 to column 6 and from column 7 to column 11 by transfer 5 μL to 45 μL DMSO to make following concentrations:

| | Final | |
|---|---|---|
| First plate | concentration | |
| Column 12 | 0 | |
| Column 11 | 0.03 | μM |
| Column 10 | 0.3 | μM |
| Column 9 | 3 | μM |
| Column 8 | 0.03 | mM |
| Column 7 | 0.3 | mM |
| Column 6 | 0.01 | μM |
| Column 5 | 0.1 | μM |
| Column 4 | 1 | μM |
| Column 3 | 0.01 | mM |
| Column 2 | 0.1 | mM |
| Column 1 | 1 | mM |

Fill a new 96-well plate with 247.5 μL of compound dilution buffer. Transfer 2.5 μL of serially diluted compounds from above plate to the new plate (1:100 dilution) as following:

| | | Final | |
|---|---|---|---|
| First plate | Second plate | concentration | |
| Column 12 | Column 1 | 0 | |
| Column 6 | Column 2 | 0.1 | nM |
| Column 11 | Column 3 | 0.3 | nM |
| Column 5 | Column 4 | 1 | nM |
| Column 10 | Column 5 | 3 | nM |
| Column 4 | Column 6 | 0.01 | μM |
| Column 9 | Column 7 | 0.03 | μM |
| Column 3 | Column 8 | 0.1 | μM |
| Column 8 | Column 9 | 0.3 | μM |
| Column 2 | Column 10 | 1 | μM |
| Column 7 | Column 11 | 3 | μM |
| Column 1 | Column 12 | 10 | μM |

### Cell Growth

1. LS174 T are always grown in MEM (ATCC Cat #30-2003), 10% FBS (ATCC Cat #30-2020) and additional 2 mM L-glutamine, at 37° C. and 5% CO₂.
2. Warm 0.05% Trypsin and Versine (Invitrogen Cat #25300-054) at 37° C. water bath.
3. Remove growth medium from cells. Cells in T165 flask are washed twice with 4 mL Trypsin followed by incubation at 37° C. and 5% CO₂ for 3 minutes.
4. Add 10 mL of medium and pipette thoroughly to separate the cells and count the cells.
5. Bring the cell density to 2.25×10⁵ cells/ml and seed 200 μL cells/well (45,000 cells/well) in 96-well plates 1 day before the assay.

### Assay Procedure

### Day 1

Seed 45,000 cells/well in 200 μL medium in 96-well plates. Incubate the cell plate at 37° C., 5% CO₂ and 95% humidity overnight.

### Day 2

1. Perform compound dilution.
2. Prepare assay buffer, lysis buffer 1 & 2, PGD₂ and standard.
3. Aspirate media from the cells and add 100 μL of compound solution using Zymark Sciclone-ALH/FD protocol cAMP DP.
4. Incubate the cells at 37° C., 5% CO₂ and 95% humidity for 15 minutes.
5. Add 5 μL of 300 nM PGD2 (20×15 nM final concentration) into each well using Zymark protocol cAMP DP PGD2, and incubate the cells at 37° C., 5% CO₂ and 95% humidity for additional 15 minutes.
6. Aspirate media from the cells and add 50 μL of lysis buffer 1 using Zymark protocol cAMP DP lysis, and incubate at room temperature with shaking for 30 minutes.
7. Add 150 μL immunoreagent to all wells (a total volume of 200 μL/well).
8. Seal the plates and shake for 2 minutes, put into the chamber of the Wallac microtitre plate μ scintillation counter for 16 hours.

### Day 3

Count the amount of [¹²⁵I] cAMP for 2 minutes in 1450 Trilux scintillation counter.

### Data Processing

Set up standard curve of cAMP versus CPM.

TABLE 1
Typical assay data for standard

| | | |
|---|---|---|
| cAMP | | Average |
| (pmol/mL) | CPM | CPM |

| | | | |
|---|---|---|---|
| 0.2 | 5725 | 5769 | 5530 |
| 0.4 | 5367 | 5259 | 6317 |
| 0.8 | 4695 | 4796 | 6507 |
| 1.6 | 4251 | 4178 | 6581 |
| 3.2 | 3434 | 3429 | 6601 |
| 6.4 | 2758 | 2716 | 6711 |
| 12.8 | 2094 | 2054 | 6680 |
| 25.6 | 1531 | 1573 | 6653 |

The cAMP concentrations (pmol/mL) of unknown samples are calculated from a standard curve of cAMP versus CPM. % inhibition is calculated using the following formula:

$$\% Inhibition = \frac{( pmol of control - pmol of sample ) \times 100}{pmol of control ( cells + PGD 2 only )}$$

The present invention may be embodied in other specific forms without departing from the spirit or essential attributes thereof.

We claim:

1. A compound of formula (I): 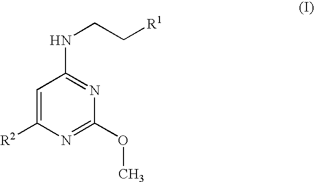
wherein:
R¹ is 2,4-dichloro-phenyl or 4-trifluoromethoxy-phenyl, and
when R¹ is 2,4-dichloro-phenyl, then R² is 3-carboxy-pyrrolidinyl, 3,5-di-(1-hydroxy-1-methyl-ethyl)-phenyl, 3-amino-piperidin-1-yl, 4-amino-piperidin-1-yl, 4-acetamide-piperidin-1-yl, 1-methyl-2-carboxy-2,3-dihydro-1H-indol-5-yl, 3-(1-tert-butylsulfonylaminocarbonyl-1-methyl-ethyl)-phenyl, 3-(1-dimethylaminosulfonylaminocarbonyl-1-methyl-ethyl)-phenyl, 3-(1-thiomorpholin-4-ylcarbonyl-1-methyl-ethyl)-phenyl, 3-(1-aminocarbonyl-1-methyl-ethyl)-phenyl, 3-(1-dimethylaminocarbonyl-1-methyl-ethyl)-phenyl, 3-carboxymethyl-piperidin-1-yl, 3-methylsulfonylaminocarbonyl-piperidin-1-yl, 3-ethylsulfonylaminocarbonyl-piperidin-1-yl, 3-tert-butylsulfonylaminocarbonyl-piperidin-1-yl, 3-trifluoromethylsulfonylaminocarbonyl-piperidin-1-yl, 3-[(1H-tetrazol-5-yl)-aminocarbonyl]-piperidin-1-yl, 3-aminocarbonyl-piperidin-1-yl, 3-dimethylaminocarbonyl-piperidin-1-yl, 3-dimethylaminosulfonylaminocarbonyl-piperidin-1-yl, or 2-carboxy-2,3-dihydro-benzofuran-5-yl, and
when R¹ is 4-trifluoromethoxy-phenyl, then R² is 3-(1-methyl-1-carboxy-ethyl)-piperidinyl, 3-carboxy-piperidinyl, 3-methylsulfonylaminocarbonyl-piperidin-1-yl, 5-carboxy-thiophen-2-yl,
or a pharmaceutically acceptable salt, hydrate, or solvate thereof, a pharmaceutically acceptable prodrug thereof, or a pharmaceutically acceptable salt, hydrate or solvate of the prodrug.

2. The compound according to claim 1, which is
1-{6-[2-(2,4-Dichloro-phenyl)-ethylamino]-2-methyl-pyrimidin-4-yl}-pyrrolidine-3-carboxylic acid,
2-(1-{2-Methoxy-6-[2-(4-trifluoromethoxy-phenyl)-ethylamino]-pyrimidin-4-yl}-piperidin-3-yl)-2-methyl-propionic acid,
2-[3-{6-[2-(2,4-Dichloro-phenyl)-ethylamino]-2-methoxy-pyrimidin-4-yl}-5-(1-hydroxy-1-methyl-ethyl)-phenyl]-propan-2-ol,
[6-(3-Amino-piperidin-1-yl)-2-methoxy-pyrimidin-4-yl]-[2-(2,4-dichloro-phenyl)-ethyl]-amine,
[6-(4-Amino-piperidin-1-yl)-2-methoxy-pyrimidin-4-yl]-[2-(2,4-dichloro-phenyl)-ethyl]-amine,
N-(1-{6-[2-(2,4-Dichloro-phenyl)-ethylamino]-2-methoxy-pyrimidin-4-yl}-piperidin-4-yl)-acetamide,
5-{6-[2-(2,4-Dichloro-phenyl)-ethylamino]-2-methoxy-pyrimidin-4-yl}-1-methyl-2,3-dihydro-1H-indole-2-carboxylic acid,
2-Methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide,
N,N-dimethylamide-2-sulfonic acid [2-(3-{6-[2-(2,4-dichloro-phenyl)-ethylamino]-2-methoxy-pyrimidin-4-yl}-phenyl)-2-methyl-propionyl]-amide,
2-(3-{6-[2-(2,4-Dichloro-phenyl)-ethylamino]-2-methoxy-pyrimidin-4-yl}-phenyl)-2-methyl-1-thiomorpholin-4-yl-propan-1-one,
2-(3-{6-[2-(2,4-Dichloro-phenyl)-ethylamino]-2-methoxy-pyrimidin-4-yl}-phenyl)-isobutyramide,
2-(3-{6-[2-(2,4-Dichloro-phenyl)-ethylamino]-2-methoxy-pyrimidin-4-yl}-phenyl)-N,N-dimethyl-isobutyramide,
(1-{6-[2-(2,4-Dichloro-phenyl)-ethylamino]-2-methoxy-pyrimidin-4-yl}-piperidin-3-yl)-acetic acid,
1-{2-Methoxy-6-[2-(4-trifluoromethoxy-phenyl)-ethylamino]-pyrimidin-4-yl}-piperidine-3-carboxylic acid,
N-(1-{2-Methoxy-6-[2-(4-trifluoromethoxy-phenyl)-ethylamino]-pyrimidin-4-yl}-piperidine-3-carbonyl)-methanesulfonamide,
N-(1-{6-[2-(2,4-Dichloro-phenyl)-ethylamino]-2-methoxy-pyrimidin-4-yl}-piperidine-3-carbonyl)-methanesulfonamide,
Ethanesulfonic acid (1-{6-[2-(2,4-dichloro-phenyl)-ethylamino]-2-methoxy-pyrimidin-4-yl}-piperidine-3-carbonyl)-amide,
2-Methyl-propane-2-sulfonic acid (1-{6-[2-(2,4-dichloro-phenyl)-ethylamino]-2-methoxy-pyrimidin-4-yl}-piperidine-3-carbonyl)-amide,
N-(1-{6-[2-(2,4-Dichloro-phenyl)-ethylamino]-2-methoxy-pyrimidin-4-yl}-piperidine-3-carbonyl)-C,C,C-trifluoro-methanesulfonamide,
1-{6-[2-(2,4-Dichloro-phenyl)-ethylamino]-2-methoxy-pyrimidin-4-yl}-piperidine-3-carboxylic acid (1H-tetrazol-5-yl)-amide,
1-{6-[2-(2,4-Dichloro-phenyl)-ethylamino]-2-methoxy-pyrimidin-4-yl}-piperidine-3-carboxylic acid amide,
1-{6-[2-(2,4-Dichloro-phenyl)-ethylamino]-2-methoxy-pyrimidin-4-yl}-piperidine-3-carboxylic acid dimethylamide,
N,N-Dimethylamide-2-sulfonic acid 1-{6-[2-(2,4-dichloro-phenyl)-ethylamino]-2-methoxy-pyrimidin-4-yl}-piperidine-3-carboxamide,
5-{2-Methoxy-6-[2-(4-trifluoromethoxy-phenyl)-ethylamino]-pyrimidin-4-yl}-thiophene-2-carboxylic acid, or
5-{6-[2-(2,4-Dichloro-phenyl)-ethylamino]-2-methoxy-pyrimidin-4-yl}-2,3-dihydro-benzofuran-2-carboxylic acid.

3. The compound or the ester prodrug according to claim 1, which is
1-{6-[2-(2,4-Dichloro-phenyl)-ethylamino]-2-methyl-pyrimidin-4-yl}-pyrrolidine-3-carboxylic acid,
2-(1-{2-Methoxy-6-[2-(4-trifluoromethoxy-phenyl)-ethylamino]-pyrimidin-4-yl}-piperidin-3-yl)-2-methyl-propionic acid,
2-[3-{6-[2-(2,4-Dichloro-phenyl)-ethylamino]-2-methoxy-pyrimidin-4-yl}-5-(1-hydroxy-1-methyl-ethyl)-phenyl]-propan-2-ol,
[6-(3-Amino-piperidin-1-yl)-2-methoxy-pyrimidin-4-yl]-[2-(2,4-dichloro-phenyl)-ethyl]-amine,
[6-(4-Amino-piperidin-1-yl)-2-methoxy-pyrimidin-4-yl]-[2-(2,4-dichloro-phenyl)-ethyl]-amine,
N-(1-{6-[2-(2,4-Dichloro-phenyl)-ethylamino]-2-methoxy-pyrimidin-4-yl}-piperidin-4-yl)-acetamide,
5-{6-[2-(2,4-Dichloro-phenyl)-ethylamino]-2-methoxy-pyrimidin-4-yl}-1-methyl-2,3-dihydro-1H-indole-2-carboxylic acid,
2-Methyl-propane-2-sulfonic acid [2-(3-{6-[2-(2,4-dichloro-phenyl)-ethylamino]-2-methoxy-pyrimidin-4-yl}-phenyl)-2-methyl-propionyl]-amide,
N,N-dimethylamide-2-sulfonic acid [2-(3-{6-[2-(2,4-dichloro-phenyl)-ethylamino]-2-methoxy-pyrimidin-4-yl}-phenyl)-2-methyl-propionyl]-amide,
2-(3-{6-[2-(2,4-Dichloro-phenyl)-ethylamino]-2-methoxy-pyrimidin-4-yl}-phenyl)-2-methyl-1-thiomorpholin-4-yl-propan-1-one,
2-(3-{6-[2-(2,4-Dichloro-phenyl)-ethylamino]-2-methoxy-pyrimidin-4-yl}-phenyl)-isobutyramide,
2-(3-{6-[2-(2,4-Dichloro-phenyl)-ethylamino]-2-methoxy-pyrimidin-4-yl}-phenyl)-N,N-dimethyl-isobutyramide,
(1-{6-[2-(2,4-Dichloro-phenyl)-ethylamino]-2-methoxy-pyrimidin-4-yl}-piperidin-3-yl)-acetic acid,
1-{2-Methoxy-6-[2-(4-trifluoromethoxy-phenyl)-ethylamino]-pyrimidin-4-yl}-piperidine-3-carboxylic acid,
N-(1-{2-Methoxy-6-[2-(4-trifluoromethoxy-phenyl)-ethylamino]-pyrimidin-4-yl}-piperidine-3-carbonyl)-methanesulfonamide,
5-{6-[2-(2,4-dichloro-phenyl)-ethylamino]-2-methoxy-pyrimidin-4-yl}-1-methyl-2,3-dihydro-1H-indole-2-carboxylic acid ethyl ester,
(1-{6-[2-(2,4-dichloro-phenyl)-ethylamino]-2-methoxy-pyrimidin-4-yl}-piperidin-3-yl)-acetic acid ethyl ester,
N-(1-{6-[2-(2,4-Dichloro-phenyl)-ethylamino]-2-methoxy-pyrimidin-4-yl}-piperidine-3-carbonyl)-methanesulfonamide,
Ethanesulfonic acid (1-{6-[2-(2,4-dichloro-phenyl)-ethylamino]-2-methoxy-pyrimidin-4-yl}-piperidine-3-carbonyl)-amide,
2-Methyl-propane-2-sulfonic acid (1-{6-[2-(2,4-dichloro-phenyl)-ethylamino]-2-methoxy-pyrimidin-4-yl}-piperidine-3-carbonyl)-amide,
N-(1-{6-[2-(2,4-Dichloro-phenyl)-ethylamino]-2-methoxy-pyrimidin-4-yl}-piperidine-3-carbonyl)-C,C,C-trifluoro-methanesulfonamide,
1-{6-[2-(2,4-Dichloro-phenyl)-ethylamino]-2-methoxy-pyrimidin-4-yl}-piperidine-3-carboxylic acid (1H-tetrazol-5-yl)-amide,
1-{6-[2-(2,4-Dichloro-phenyl)-ethylamino]-2-methoxy-pyrimidin-4-yl}-piperidine-3-carboxylic acid amide,
1-{6-[2-(2,4-Dichloro-phenyl)-ethylamino]-2-methoxy-pyrimidin-4-yl}-piperidine-3-carboxylic acid dimethylamide,
N,N-Dimethylamide-2-sulfonic acid 1-{6-[2-(2,4-dichloro-phenyl)-ethylamino]-2-methoxy-pyrimidin-4-yl}-piperidine-3-carboxamide,
5-{2-Methoxy-6-[2-(4-trifluoromethoxy-phenyl)-ethylamino]-pyrimidin-4-yl}-thiophene-2-carboxylic acid, or
5-{6-[2-(2,4-Dichloro-phenyl)-ethylamino]-2-methoxy-pyrimidin-4-yl}-2,3-dihydro-benzofuran-2-carboxylic acid,
or a pharmaceutically acceptable salt, hydrate, or solvate thereof.

4. A pharmaceutical composition comprising a pharmaceutically effective amount of the compound according to claim 1 or a pharmaceutically acceptable salt, hydrate, or solvate thereof, a pharmaceutically acceptable prodrug thereof, or a pharmaceutically acceptable salt, hydrate or solvate of the prodrug, in admixture with a pharmaceutically acceptable carrier.

5. A method for treating an allergic disease, systemic mastocytosis, a disorder accompanied by systemic mast cell activation, anaphylaxis shock, bronchoconstriction, bronchitis, urticaria, eczema, a diseases accompanied by itch, a disease which is generated secondarily as a result of behavior accompanied by itch, inflammation, chronic obstructive pulmonary diseases, ischemic reperfusion injury, cerebrovascular accident, chronic rheumatoid arthritis, pleurisy, or ulcerative colitis, in a patient in need thereof, comprising administering to the patient a pharmaceutically effective amount of the compound according to claim 1, or a pharmaceutically acceptable salt, hydrate, or solvate thereof, a pharmaceutically acceptable prodrug thereof, or a pharmaceutically acceptable salt, hydrate or solvate of the prodrug.

6. The method according to claim 5, wherein the a disease which is generated secondarily as a result of behavior accompanied by itch is cataract, retinal detachment, inflammation, infection or sleeping disorder.

7. The method according to claim 5, wherein the allergic disease is allergic rhinitis, allergic conjunctivitis, atopic dermatitis, bronchial asthma or food allergy.

8. The method according to claim 5, wherein the disease accompanied by itch is atopic dermatitis or urticaria.

9. The method according to claim 5, wherein the disease that is generated secondarily as a result of behavior accompanied by itch is cataract, retinal detachment, inflammation, infection or sleeping disorder.

10. The method according to claim 5, which is for treating bronchial asthma.

11. The method according to claim 5, which is for treating allergic rhinitis.

12. The method according to claim 5, which is for treating allergic dermatitis.

13. The method according to claim 5, which is for treating allergic conjunctivitis.

14. The method according to claim 5, which is for treating chronic obstructive pulmonary disease.

15. A pharmaceutical composition comprising a pharmaceutically effective amount of the compound according to claim 1 or a pharmaceutically acceptable salt, hydrate, or solvate thereof, a pharmaceutically acceptable prodrug thereof, or a pharmaceutically acceptable salt, hydrate or solvate of the prodrug, and a compound selected from the group consisting of an antihistamine, a leukotriene antagonist, a beta agonist, a PDE4 inhibitor, a TP antagonist and a CrTh2 antagonist, in admixture with a pharmaceutically acceptable carrier.

16. The pharmaceutical composition according to claim 15, wherein the antihistamine is fexofenadine, loratadine or citirizine, the leukotriene antagonist is montelukast or zafirlukast, the beta agonist is albuterol, salbuterol or terbutaline, the PDE4 inhibitor is roflumilast or cilomilast, the TP antagonist is Ramatroban, and the CrTh2 antagonist is Ramatroban.
